(19) United States
(12) Patent Application Publication
(10) Pub. No.: US 2022/0096543 A1
(43) Pub. Date: Mar. 31, 2022

(54) TOLLIP DEFICIENT NEUTROPHILS AND USES THEREOF

(71) Applicant: VIRGINA TECH INTELLECTUAL PROPERTIES, INC., BLACKSBURG, VA (US)

(72) Inventors: Liwu LI, Blacksburg, VA (US); Yao ZHANG, Blacksburg, VA (US); Christina LEE, Blacksburg, VA (US)

(21) Appl. No.: 17/418,037

(22) PCT Filed: Dec. 23, 2019
(86) PCT No.: PCT/US2019/068443

§ 371 Date: Jun. 24, 2021

Related U.S. Application Data
(60) Provisional application No. 62/784,595, filed on Dec. 24, 2018.

(51) Int. Cl. A61K 35/15; C12N 15/90; A61K 31/7088; A61P 35/00

(52) U.S. Cl. CPC A61K 35/15; A61P 35/00; A61K 31/7088; C12N 15/907

(57) ABSTRACT

Described herein are modified cells and compositions thereof, wherein the cells can have reduced or eliminated Tollip gene and/or protein expression. In some embodiments, the modified cells can be neutrophils. Also described herein are methods of making and using the modified cells and compositions thereof. In some embodiments, the modified cells having reduced or eliminated Tollip gene and/or protein expression can be administered to a subject in need thereof.

### CROSS REFERENCE TO RELATED APPLICATIONS

This application claims the benefit of and priority to co-pending U.S. Provisional Patent Application No. 62/784,595, filed on Dec. 24, 2018, entitled “TOLLIP DEFICIENT NEUTROPHILS AND USES THEREOF,” the contents of which is incorporated by reference herein in its entirety.

### STATEMENT REGARDING FEDERALLY SPONSORED RESEARCH

This invention was made with Government support AI124673 awarded by the National Institutes of Health. The Government has certain rights in the invention.

### TECHNICAL FIELD

The subject matter disclosed herein is generally directed to modified immune cells, particularly neutrophils.

### BACKGROUND

Cancer is a collection of related diseases characterized by abnormal cell growth that has the potential to spread to other parts of the body. Cancer is significant health issue worldwide. In the United States, it is estimated that in 2018 there will be about 1.7 million new cases diagnosed and about 600,000 people will die from the disease. Alarmingly, it is estimated that 38.4% of people will be diagnosed with cancer at some point during their life. Although significant advances have been made in cancer treatment and prevention, there still exists a need for additional treatments and prevention for cancer.

### SUMMARY

In some exemplary embodiments, described herein is a modified neutrophil or population thereof having reduced or eliminated Tollip gene expression and/or amount of Tollip protein as compared to a wild-type or suitable control neutrophil. In some exemplary embodiments, the modified neutrophil comprises a deletion of one or more copies of the Tollip gene. In some exemplary embodiments, the modified neutrophil comprises a Tollip gene silencing oligonucleotide. In some exemplary embodiments, the modified neutrophil comprises a suicide gene. In some exemplary embodiments, the modified neutrophil has increased gene and/or protein expression of CD80 as compared to a wild-type neutrophil or suitable control. In some exemplary embodiments, the modified neutrophil has decreased gene and/or protein expression of PDL-las compared to a wild-type neutrophil or a suitable control. In some exemplary embodiments, the modified neutrophil is a human neutrophil.

In some exemplary embodiments, described herein are pharmaceutical formulations comprising a modified neutrophil as is described anywhere herein or a population thereof; and a pharmaceutically acceptable carrier. In some exemplary embodiments, the pharmaceutical formulation comprises a therapeutically effective amount of the modified neutrophil or population thereof.

In some exemplary embodiments, described herein are methods of generating a Tollip deficient neutrophil or population thereof, that can include: harvesting neutrophils from a subject to obtain harvested neutrophils; deleting one or more copies of the Tollip gene in one or more of the harvested neutrophils in vitro to obtain the Tollip deficient neutrophil or population thereof. In some exemplary embodiments, the Tollip deficient neutrophil or population thereof has reduced or eliminated Tollip gene expression and/or amount of Tollip protein as compared to a wild-type or suitable control neutrophil. In some exemplary embodiments, the Tollip deficient neutrophil or population thereof has increased gene and/or protein expression of CD80 as compared to a wild-type neutrophil or suitable control. In some exemplary embodiments, the Tollip deficient neutrophil or population thereof has decreased gene and/or protein expression of PDL-1 as compared to a wild-type neutrophil or suitable control. In some exemplary embodiments, the method further comprises the step of transforming a harvested neutrophil or the Tollip deficient neutrophil or population thereof to contain and/or conditionally express a suicide gene. In some exemplary embodiments, the subject is a human.

In some exemplary embodiments, method can further include the step of administering the Tollip deficient neutrophil or population thereof to a subject in need thereof. In some exemplary embodiments, the subject and the subject in need thereof are the same. In some exemplary embodiments, the subject and the subject in need thereof are the different.

In some exemplary embodiments, described herein are methods of generating a Tollip deficient neutrophil or population thereof, the method comprising: transforming a neutrophil with a Tollip gene silencing oligonucleotide to generate the Tollip deficient neutrophil or population thereof. In some exemplary embodiments, the method can further include the step of transforming a neutrophil with a suicide gene.

In some exemplary embodiments, the method further includes harvesting neutrophils from a subject to obtain harvested neutrophils and wherein the one or more of the harvested neutrophils are transformed in vitro to generate a Tollip deficient neutrophil or population thereof.

In some exemplary embodiments, the method can further include administering the Tollip deficient neutrophil or population thereof to a subject in need thereof. In some exemplary embodiments, the subject and the subject in need thereof are the same. In some exemplary embodiments, the subject and the subject in need thereof are different. In some exemplary embodiments, the subject and the subject in need thereof are human.

In some exemplary embodiments, the method can further include administering a Tollip gene silencing oligonucleotide to a subject in need thereof. In some exemplary embodiments, the step of transformation occurs in vivo. In some exemplary embodiments, the subject in need thereof is human. In some exemplary embodiments, the Tollip deficient neutrophil or population thereof has reduced or eliminated Tollip gene expression and/or amount of Tollip protein as compared to a wild-type or suitable control neutrophil. In some exemplary embodiments, the Tollip deficient neutrophil or population thereof has increased gene and/or protein expression of CD80 as compared to a wild-type neutrophil or suitable control. In some exemplary embodiments, the Tollip deficient neutrophil or population thereof has decreased gene and/or protein expression of PDL-las compared to a wild-type neutrophil or suitable control.

In some exemplary embodiments, described herein are methods that can include administering a modified neutrophil or population thereof as described anywhere herein to a subject. In some exemplary embodiments, the subject is a subject in need thereof and has or is suspected of having a cancer.

In some exemplary embodiments, described herein are methods of administering a pharmaceutical formulation as described anywhere herein to a subject. In some exemplary embodiments, the subject is a subject in need thereof and has or is suspected of having a cancer.

In some exemplary embodiments, described herein are methods of treating and/or preventing cancer in a subject in need thereof, the method comprising: administering a modified neutrophil or population thereof as described anywhere herein to the subject in need thereof.

In some exemplary embodiments, described herein are methods of treating and/or preventing cancer in a subject in need thereof, the method comprising: administering a pharmaceutical formulation as described anywhere herein to the subject in need thereof.

In some exemplary embodiments, described herein are methods of treating and/or preventing cancer in a subject in need thereof, the method comprising: administering a Tollip gene silencing oligonucleotide to a subject in need thereof.

### BRIEF DESCRIPTION OF THE DRAWINGS

Further aspects of the present disclosure will be readily appreciated upon review of the detailed description of its various embodiments, described below, when taken in conjunction with the accompanying drawings.

FIGS. 1A-1H show schematics, images, and graphs that can demonstrate Tollip deficiency decreased tumorigenesis in the AOM-DSS mouse model of colon cancer. (FIG. 1A) Schematic protocol of AOM-DSS treatment. (FIG. 1B) Representative images of colons from WT and Tollip−/− mice treated with AOM-DSS or naïve mice. (FIG. 1C) Graphical representation of tumor burden in WT (n=6) and Tollip−/− (n=8) mice. Diameter of tumors greater than or equal to 2 mm defined as “macro” tumor; diameter of tumors less than 2 mm defined as “micro” tumor. (FIG. 1D) H&E stained sections of colon from WT or Tollip−/− mice treated with AOM-DSS treated or naïve mice. Colons were collected in swiss rolls at the end of AOM-DSS regimen. Scale bar represents 2.0 mm. (FIG. 1E) Immunofluorescent analysis of Ki67 in colons of from WT or Tollip−/− mice treated with AOM-DSS treated or naïve mice. Scale bar represents 200 μm. (FIG. 1F) Quantitative analysis of Ki67 staining. (FIG. 1G) Immunofluorescent analysis of active β-catenin in colons from WT or Tollip−/− mice treated with AOM-DSS treated or naïve mice. Scale bar represents 200 μm. (FIG. 1H) Quantitative analysis of active β-catenin staining. * p<0.05, ** p<0.01, *** p<0.001.

FIGS. 2A-2E can demonstrate that Tollip deficiency enhanced anti-tumor innate immune checkpoints. (FIG. 2A) PD-L1 and CD80 expression on the neutrophils in the spleens from WT or Tollip−/− mice with AOM-DSS treatment or naïve mice. (FIG. 2B) Percentages of CD4+ and CD8+ cells in the colon lamina propria from WT or Tollip−/− mice with AOM-DSS treatment or naïve mice. (FIG. 2C) Cytokine profiles of colons collected from WT or Tollip−/− mice treated with AOM-DSS. (FIG. 2D) Cytokine profiles of plasma collected from WT or Tollip−/− mice treated with AOM-DSS. (FIG. 2E) CD14 and CCR5 expression on the surface of neutrophils in the blood. * p<0.05, ** p<0.01, *** p<0.001.

FIGS. 3A-3D can demonstrate that Tollip deficiency released the neutrophil suppression on T cell proliferation viaPD-L1/CD80. (FIG. 3A) CFSE-labeled splenocytes were cocultured with GM-CSF primed neutrophils in the anti-CD3 antibody coated plates for 72 hours. Representative results are shown. (FIG. 3B) PD-L1 and CD80 expression on GM-CSF primed neutrophils. * p<0.05, ** p<0.01. (FIG. 3C) In the presence of anti-PD-L1 antibody, CFSE-labeled splenocytes were cocultured with GM-CSF primed WT neutrophils in the anti-CD3 antibody coated plates for 72 hours. (FIG. 3D) In the presence of anti-CD80 antibody, CFSE-labeled splenocytes were cocultured with GM-CSF primed WT neutrophils in the anti-CD3 antibody coated plates for 72 hours.

FIGS. 4A-4E can demonstrate that Tollip−/− neutrophils facilitated T cell activation and survival. (FIG. 4A) (FIG. 4A) Splenocytes were cocultured with GM-CSF-primed neutrophils (WT or Tollip−/−) in anti-CD3 antibody-coated plates for 24 hours, and then CD62L levels on CD4+ or CD8+ T cells were measured by flow cytometry. Representative results are shown. (FIG. 4B) After coculture, CD69 levels on CD4+ cells and CD107a+ cells in CD8+ cells were analyzed. (FIG. 4C) Conditional medium from coculture was analyzed by ELISA. (FIG. 4D) Splenocytes were cocultured with GM-CSF-primed neutrophils (WT or Tollip−/−) for 72 hours, before cell viabilities were tested. (FIG. 4E) Quantification analysis of the cell viabilities. Statistical significance compared with WT in the same treatment conditions was determined by Mann-Whitney U test (FIGS. 4B and 4C) or Student's t test (FIG. 4E). *P<0.05, **P<0.01, ***P<0.01.

FIGS. 5A-5C can demonstrate Tollip deficiency released the neutrophil suppression on T cell proliferation via PD-L1/CD80 signaling pathway. (FIG. 5A) Immunoblotting analysis of STAT1, STAT3 and IRF1 in lysates from fresh bone marrow neutrophils or neutrophils primed with GM-CSF overnight. (FIG. 5B) Immunoblotting analysis of STAT5, p65, and oxCaMKII in lysates from fresh bone marrow neutrophils or neutrophils primed with GM-CSF overnight. (FIG. 5C) Flow cytometry analysis of phosho-proteins in fresh bone marrow neutrophils or neutrophils primed with GM-CSF overnight, pre-gated on Ly6G+ cells. * p<0.05.

FIGS. 6A-6F can demonstrate that adoptive transfer of Tollip−/− neutrophils to WT mice slows down colitis-associated cancer progression. (FIG. 6A) Representative images of colons from WT mice received WT or Tollip−/− neutrophils on Day 64. (FIG. 6B) Graphical representation of tumor burden in WT mice received WT or Tollip−/− neutrophils. N≥5 each group. (FIG. 6C) H&E stained sections of colon from the mice received WT or Tollip−/− neutrophils. Colons were collected in swiss rolls at the end of AOM-DSS regimen. Scale bar represent 2.5 mm (top) and 0.5 mm (bottom). (FIG. 6D) Immunofluorescent analysis of Ki67 and β-catenin. Blue color is DAPI staining. Scale bar represents 200 μm. (FIG. 6E) CD4+ and CD8+ cell counts in the spleens from the mice received WT or Tollip−/− neutrophils. (FIG. 6F) Percentages of CD62L low in CD8+ T cells. Percentage of Granzyme B positive cells in CD8+ T cells. * p<0.05, ** p<0.01, *** p<0.001.

FIG. 7 shows a graph that can demonstrate the survival curves of WT and Tollip−/− mice treated with AOM-DSS treatment. N=5 each group.

FIG. 8 shows a graph that can demonstrate the body weight change of wild-type and WT and Tollip−/− mice treated with AOM-DSS treatment. The body weight change curves of WT and Tollip−/− mice during AOM-DSS treatment. N=5 each group, and values were expressed as means.

FIG. 9 shows a graph that can demonstrate stool clinical evaluations of wild-type and WT and Tollip−/− mice treated with AOM-DSS treatment. Stool clinical scores including stool consistency and bleeding of WT and Tollip−/− mice. N=8, values were expressed as means.

FIG. 10 shows a graph that can demonstrate a comparison of stool clinical scores. Stool clinical scores including stool consistency and bleeding of WT and Tollip−/− mice were collected and compared at the end of each cycle (rest day 14). N=8, values were expressed as means±SD. * p<0.05.

FIGS. 11A-11B show graphs that can demonstrate elevated T cell population was observed in Tollip deficiency mice. (FIG. 11A) Neutrophil (Ly6G+CD11b+) percentages in the blood and colon from naïve WT and Tollip−/− mice, or AOM-DSS treated WT and Tollip−/− mice. (FIG. 11B) CD4+ and CD8+ cell counts in the spleens from WT or Tollip−/− mice with AOM-DSS treatment or naïve mice.

FIGS. 12A-12D can show (FIG. 12A) The schematic protocol of AOM-DSS treatment with adoptive transfer (A.T.) of WT or Tollip−/− neutrophils to WT mice. (FIG. 12B) Colon length from mice received WT or Tollip−/− neutrophils at the end of AOM-DSS regimen. (FIG. 12C) Body weight change curves of the mice transferred with WT or Tollip−/− neutrophils during AOM-DSS treatment. (FIG. 12D) Stool clinical scores including stool consistency and bleeding of the mice transferred with WT or Tollip−/− neutrophils.

FIGS. 13A-13C can show results from examination of immune cells in mice treated with AOM-DSS. Percentages and surface molecules of B cells (FIG. 13A), T cells (FIG. 13B), and monocytes (FIG. 13C) in the spleen from AOM-DSS treated WT and Tollip−/− mice.

FIG. 14 can demonstrate reduced CD14 expression on Tollip deficient neutrophils. CD14 expression on neutrophils from spleen or colon in naïve WT and Tollip−/− mice, or AOM-DSS treated WT and Tollip−/− mice was examined by flow cytometry. * p<0.05.

FIGS. 15A-15B can demonstrate the modulation of T cell activation by neutrophils through PDL1-CD80. In the presence of anti-PD-L1 or anti-CD80 antibodies, splenocytes were co-cultured with GM-CSF primed neutrophils in the anti-CD3 antibody coated plates for 24 hours, then CD69 levels on CD4+ T cells were measured by flow cytometry (A). CD107a positive cells were analyzed in CD8+ cells (B). * p<0.05; ** p<0.01.

FIGS. 16A-16E can demonstrate adoptive transfer of Tollip−/− monocytes to WT mice. (FIG. 16A) Schematic protocol of AOM-DSS treatment with adoptive transfer (A.T.) of WT or Tollip−/− monocytes to WT mice. (FIG. 16B) Tumor burden in WT mice which received WT or Tollip−/− monocytes. (FIG. 16C) Colon length at the end of AOM-DSS regimen from mice which received WT or Tollip−/− monocytes. (FIG. 16D) Body weight change curves of the mice which received WT or Tollip−/− monocytes during AOM-DSS treatment. (FIG. 16E) Stool clinical scores including stool consistency and bleeding of the mice which received WT or Tollip−/− monocytes. *** p<0.01.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used in the specification and the appended claims, the singular forms “a,” “an,” and “the” include plural referents unless the context clearly dictates otherwise.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Embodiments of the present disclosure will employ, unless otherwise indicated, techniques of molecular biology, microbiology, organic chemistry, biochemistry, physiology, cell biology, cancer biology, and the like, which are within the skill of the art. Such techniques are explained fully in the literature.

Definitions of common terms and techniques in molecular biology may be found in Molecular Cloning: A Laboratory Manual, 2_{nd} edition (1989) (Sambrook, Fritsch, and Maniatis); Molecular Cloning: A Laboratory Manual, 4ₜₕ edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2_{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2_{nd} edition (2011).

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible unless the context clearly dictates otherwise.

### Definitions

As used herein, “active agent” or “active ingredient” can refer to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques.

As used herein, “agent” can refer to any substance, compound, molecule, and the like, which can be biologically active or otherwise can induce a biological and/or physiological effect on a subject to which it is administered to. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein “cancer” refers to one or more types of cancer including, but not limited to, acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, astrocytomas, atypical teratoid/Rhabdoid tumors, basa cell carcinoma of the skin, bile duct cancer, bladder cancer, bone cancer (including but not limited to Ewing Sarcoma, osteosarcomas, and malignant fibrous histiocytoma), brain tumors, breast cancer, bronchial tumors, Burkitt lymphoma, carcinoid tumor, cardiac tumors, germ cell tumors, embryonal tumors, cervical cancer, cholangiocarcinoma, chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, endometrial cancer, ependymoma, esophageal cancer, esthesioneuroblastoma, extracranial germ cell tumor, extragonadal germ cell tumor, eye cancer (including, but not limited to, intraocular melanoma and retinoblastoma), fallopian tube cancer, gallbladder cancer, gastric cancer, gastrointestinal carcinoid tumor, gastrointestinal stromal tumors, central nervous system germ cell tumors, extracranial germ cell tumors, extragonadal germ cell tumors, ovarian germ cell tumors, testicular cancer, gestational trophoblastic disease, hary cell leukemia, head and neck cancers, hepatocellular (liver) cancer, Langerhans cell histiocytosis, Hodgkin lymphoma, hypopharyngeal cancer, islet cell tumors, pancreatic neuroendocrine tumors, kidney (renal cell) cancer, laryngeal cancer, leukemia, lip cancer, oral cancer, lung cancer (non-small cell and small cell), lymphoma, melanoma, Merkel cell carcinoma, mesothelioma, metastatic squamous cell neck cancer, midline tract carcinoma with and without NUT gene changes, multiple endocrine neoplasia syndromes, multiple myeloma, plasma cell neoplasms, mycosis fungoides, myelodyspastic syndromes, myelodysplastic/myeloproliferative neoplasms, chronic myelogenous leukemia, nasal cancer, sinus cancer, non-Hodgkin lymphoma, pancreatic cancer, paraganglioma, paranasal sinus cancer, parathyroid cancer, penile cancer, pharyngeal cancer, pheochromocytoma, pituitary cancer, peritoneal cancer, prostate cancer, rectal cancer, Rhabdomyosarcoma, salivary gland cancer, uterine sarcoma, Sezary syndrome, skin cancer, small intestine cancer, large intestine cancer (colon cancer), soft tissue sarcoma, T-cell lymphoma, throat cancer, oropharyngeal cancer, nasopharyngeal cancer, hypoharyngeal cancer, thymoma, thymic carcinoma, thyroid cancer, transitional cell cancer of the renal pelvis and ureter, urethral cancer, uterine cancer, vaginal cancer, cervical cancer, vascular tumors and cancer, vulvar cancer, and Wilms Tumor.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

As used herein, “control” can refer to an alternative subject or sample used in an experiment for comparison purpose and included to minimize or distinguish the effect of variables other than an independent variable. A “suitable control” is one that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed.

As used herein with reference to the relationship between DNA, cDNA, cRNA, RNA, protein/peptides, and the like “corresponding to” refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “deoxyribonucleic acid (DNA)” and “ribonucleic acid (RNA)” generally refers to any polyribonucleotide or polydeoxribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA).

As used herein, “differentially expressed,” refers to the differential production of RNA, including but not limited to mRNA, tRNA, miRNA, siRNA, snRNA, and piRNA transcribed from a gene or regulatory region of a genome or the protein product encoded by a gene as compared to the level of production of RNA or protein by the same gene or regulator region in a normal or a control cell. In another context, “differentially expressed,” also refers to nucleotide sequences or proteins in a cell or tissue which have different temporal and/or spatial expression profiles as compared to a normal or control cell.

As used herein, “DNA molecule” can include nucleic acids/polynucleotides that are made of DNA.

As used herein, “effective amount” refers to the amount of a compound provided herein that is sufficient to effect beneficial or desired biological, emotional, medical, or clinical response of a cell, tissue, system, animal, or human. An effective amount can be administered in one or more administrations, applications, or dosages. The term cam also include within its scope amounts effective to enhance or restore to substantially normal physiological function. The “effective amount” can refer to the amount of a modified neutrophil as described herein that can be effective to reduce tumor size, tumor number, tumor grade, cancer, or a symptom thereof.

As used herein, the term “encode” can refer to principle that DNA can be transcribed into RNA, which can then be translated into amino acid sequences that can form proteins.

As used herein, “expression” can refer to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used herein, the terms “guide polynucleotide,” “guide sequence,” or “guide RNA” as can refer to any polynucleotide sequence having sufficient complementarity with a target polynucleotide sequence to hybridize with the target sequence and direct sequence-specific binding of a CRISPR complex to the target sequence. The degree of complementarity between a guide polynucleotide and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). A guide polynucleotide (also referred to herein as a guide sequence and includes single guide sequences (sgRNA)) can be about or more than about 5, 10, 11, 12, 13, 14, 15, 16, 17, 18, 19, 20, 21, 22, 23, 24, 25, 26, 27, 28, 29, 30, 35, 40, 45, 50, 75, 90, 100, 110, 112, 115, 120, 130, 140, or more nucleotides in length. The guide polynucleotide can include a nucleotide sequence that is complementary to a target DNA sequence. This portion of the guide sequence can be referred to as the complementary region of the guide RNA. In some contexts, the two are distinguished from one another by calling one the complementary region or target region and the rest of the polynucleotide the guide sequence or tracrRNA. The guide sequence can also include one or more miRNA target sequences coupled to the 3′ end of the guide sequence. The guide sequence can include one or more MS2 RNA aptamers incorporated within the portion of the guide strand that is not the complementary portion. As used herein the term guide sequence can include any specially modified guide sequences, including but not limited to those configured for use in synergistic activation mediator (SAM) implemented CRISPR (Nature 517, 583-588 (29 Jan. 2015) or suppression (Cell Volume 154, Issue 2, 18 Jul. 2013, Pages 442-451). A guide polynucleotide can be less than about 150, 125, 75, 50, 45, 40, 35, 30, 25, 20, 15, 12, or fewer nucleotides in length. The ability of a guide polynucleotide to direct sequence-specific binding of a CRISPR complex to a target sequence may be assessed by any suitable assay. For example, the components of a CRISPR system sufficient to form a CRISPR complex, including the guide polynucleotide to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the guide polynucleotide to be tested and a control guide polynucleotide different from the test guide polynucleotide, and comparing binding or rate of cleavage at the target sequence between the test and control guide polynucleotide reactions. Other assays are possible, and will occur to those skilled in the art.

A complementary region of the gRNA can be configured to target any DNA region of interest. The complementary region of the gRNA and the gRNA can be designed using a suitable gRNA design tool. Suitable tools are known in the art and are available to the skilled artisan. As such, the constructs described herein are enabled for any desired target DNA so long as it is CRISPR compatible according to the known requirements for CRISPR activation.

A guide polynucleotide can be selected to reduce the degree of secondary structure within the guide polynucleotide. Secondary structure may be determined by any suitable polynucleotide folding algorithm. Some programs are based on calculating the minimal Gibbs free energy. An example of one such algorithm is mFold, as described by Zuker & Stiegler ((1981) Nucleic Acids Res. 9, 133-148). Another example folding algorithm is the online webserver RNAfold, developed at Institute for Theoretical Chemistry at the University of Vienna, using the centroid structure prediction algorithm (see e.g. Gruber et al., (2008) Cell 106: 23-24; and Carr & Church (2009) Nature Biotechnol. 27: 1151-1162).

As used herein, the term “homology-directed repair (HDR)” can refer to a mechanism in cells to repair double-stranded and single stranded DNA breaks. Homology-directed repair includes homologous recombination (HR) and single-strand annealing (SSA) (Lieber. (2010) Annu. Rev. Biochem. 79: 181-211). The most common form of HDR is called homologous recombination (HR), which has the longest sequence homology requirements between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks.

As used herein, “identity,” can refer to a relationship between two or more nucleotide or polypeptide sequences, as determined by comparing the sequences. In the art, “identity” can also refer to the degree of sequence relatedness between nucleotide or polypeptide sequences as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M, and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H, and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453) algorithm (e.g., NBLAST, and XBLAST). The default parameters are used to determine the identity for the polypeptides of the present disclosure, unless stated otherwise.

As used herein, “microRNA” refers to a small non-coding RNA molecule containing about 21 to about 23 nucleotides found in organisms, which functions in transcriptional and post-transcriptional regulation of transcription and translation of RNA. “MicroRNA” can exist as part of a larger nucleic acid molecule such as a stem-loop structure that can be processed by a cell and yield a microRNA of about 21-23 nucleotides.

As used herein “miRNA target” or “miRNA target sequence” refers to the nucleic acid sequence, typically RNA, that a miRNA specifically binds to. The miRNA target can be or include a sequence that is complementary to the miRNA. As an example, microRNA 126 (miR-126) can specifically bind a miR-126 target. Binding of a miRNA to a miRNA target can result in transcription and/or translation inhibition of the nucleic acid sequence, such as through degradation of the nucleic acid sequence (typically mRNA or other type of RNA), that the miRNA target is part of). A micro RNA does not have to have perfect complementarity to a miRNA target for specific binding or transcription inhibition to occur.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “negative control” refers to a “control” that is designed to produce no effect or result, provided that all reagents are functioning properly and that the experiment is properly conducted. Other terms that are interchangeable with “negative control” include “sham,” “placebo,” and “mock.”

As used herein, “nucleic acid,” “nucleotide sequence,” and “polynucleotide” can be used interchangeably herein and generally refers to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “operatively linked” in the context of recombinant DNA molecules, vectors, and the like refers to the regulatory and other sequences useful for expression, stabilization, replication, and the like of the coding and transcribed non-coding sequences of a nucleic acid that are placed in the nucleic acid molecule in the appropriate positions relative to the coding sequence so as to effect expression or other characteristic of the coding sequence or transcribed non-coding sequence. This same term can be applied to the arrangement of coding sequences, non-coding and/or transcription control elements (e.g. promoters, enhancers, and termination elements), and/or selectable markers in an expression vector. “Operatively linked” can also refer to an indirect attachment (i.e. not a direct fusion) of two or more polynucleotide sequences or polypeptides to each other via a linking molecule (also referred to herein as a linker).

As used herein, “overexpressed” or “overexpression” refers to an increased expression level of an RNA and/or protein product encoded by a gene as compared to the level of expression of the RNA or protein product in a normal or control cell. The amount of increased expression as compared to a normal or control cell can be about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, 2.0, 2.2, 2.4, 2.6, 2.8, 3.0, 3.3, 3.6, 3.9, 4.0, 4.4, 4.8, 5.0, 5.5, 6, 6.5, 7, 7.5, 8.0, 8.5, 9, 9.5, 10, 11, 12, 13, 14, 15, 16, 17, 18, 19, 20, 25, 30, 35, 40, 45, 50, 60, 70, 80, 90, 100 fold or more greater than the normal or control cell.

As used herein, “patient” refers to an organism, host, or subject in need of treatment.

As used herein “peptide” refers to chains of at least 2 amino acids that are short, relative to a protein or polypeptide.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” refers to any acid or base addition salt whose counter-ions are non-toxic to the subject to which they are administered in pharmaceutical doses of the salts.

As used herein, “plasmid” refers to a non-chromosomal double-stranded DNA sequence including an intact “replicon” such that the plasmid is replicated in a host cell.

As used herein, “positive control” refers to a “control” that is designed to produce the desired result, provided that all reagents are functioning properly and that the experiment is properly conducted.

As used herein, “preventative” and “prevent” refers to hindering or stopping a disease or condition before it occurs, even if undiagnosed, or while the disease or condition is still in the sub-clinical phase.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

As used herein, “promoter” includes all sequences capable of driving transcription of a coding or a non-coding sequence. In particular, the term “promoter” as used herein refers to a DNA sequence generally described as the 5′ regulator region of a gene, located proximal to the start codon. The transcription of an adjacent coding sequence(s) is initiated at the promoter region. The term “promoter” also includes fragments of a promoter that are functional in initiating transcription of the gene.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, “seed sequence” or “seed region” refers to a 7-nucleotide long region within a microRNA that can be conserved between 2 or more microRNAs that is typically located from nucleotides 2-7 from the 5′ end of the mature microRNA.

As used herein, the term “specific binding” can refer to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10₋₃ M or less, 10₋₄ M or less, 10₋₅ M or less, 10₋₆ M or less, 10₋₇ M or less, 10₋₈ M or less, 10₋₉ M or less, 10₋₁₀ M or less, 10₋₁₁ M or less, or 10₋₁₂ M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10₋₃ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, the terms “sufficient” and “effective,” can refer to an amount (e.g. mass, volume, dosage, concentration, and/or time period) needed to achieve one or more desired result(s). For example, a therapeutically effective amount refers to an amount needed to achieve one or more therapeutic effects.

A “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a cancer. The effect can be therapeutic in terms of a partial or complete cure of a disease, condition, symptom or adverse effect attributed to the disease, disorder, or condition. The term “treatment” as used herein covers any treatment of cancer, in a subject, particularly a human, and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, “underexpressed” or “underexpression” can refer to decreased expression level of an RNA (coding or non-coding RNA) or protein product encoded by a gene as compared to the level of expression of the RNA or protein product in a normal or control cell. The amount of decreased expression as compared to a normal or control cell can be about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, 2.0, 2.2, 2.4, 2.6, 2.8, 3.0, 3.3, 3.6, 3.9, 4.0, 4.4, 4.8, 5.0, 5.5, 6, 6.5, 7, 7.5, 8.0, 8.5, 9, 9.5, 10, 11, 12, 13, 14, 15, 16, 17, 18, 19, 20, 25, 30, 35, 40, 45, 50, 60, 70, 0, 90, 100 fold or more less than the normal or control cell.

As used herein, “variant” can refer to a polynucleotide or polypeptide that differs from a reference polynucleotide or polypeptide, but retains essential and/or characteristic properties (structural and/or functional) of the reference polynucleotide or polypeptide. A typical variant of a polypeptide differs in amino acid sequence from another, reference polypeptide. The differences can be limited so that the sequences of the reference polypeptide and the variant are closely similar overall and, in many regions, identical. A variant and reference polypeptide may differ in nucleic or amino acid sequence by one or more modifications at the sequence level or post-transcriptional or post-translational modifications (e.g., substitutions, additions, deletions, methylation, glycosylations, etc.). A substituted nucleic acid may or may not be an unmodified nucleic acid of adenine, thiamine, guanine, cytosine, uracil, including any chemically, enzymatically or metabolically modified forms of these or other nucleotides. A substituted amino acid residue may or may not be one encoded by the genetic code. A variant of a polypeptide may be naturally occurring such as an allelic variant, or it may be a variant that is not known to occur naturally. “Variant” includes functional and structural variants.

As used herein, the term “vector” or is used in reference to a vehicle used to introduce an exogenous nucleic acid sequence into a cell. A vector may include a DNA molecule, linear or circular (e.g. plasmids), which includes a segment encoding a polypeptide of interest operatively linked to additional segments that provide for its transcription and translation upon introduction into a host cell or host cell organelles. Such additional segments may include promoter and terminator sequences, and may also include one or more origins of replication, one or more selectable markers, an enhancer, a polyadenylation signal, etc. Expression vectors are generally derived from yeast or bacterial genomic or plasmid DNA, or viral DNA, or may contain elements of both. Suitable vectors, including expression vectors, are generally known in the art and will be appreciated by one of ordinary skill in the art in view of this disclosure.

As used herein, “wild-type” refers to the typical or average from of a gene, protein, species, organism, etc. as it occurs in a given population.

As used herein, “transforming” when used in the context of engineering or modifying a cell, refers to the introduction by any suitable technique and/or the transient or stable incorporation and/or expression of an exogenous gene in a cell.

As used herein, “suicide gene” refers to a gene that encodes one or more proteins that can result in apoptosis of that cell and can be inducible upon administration or contact with an exogenous molecule or agent so as to provide exogenously controlled apoptosis of a cell that carries one or more suicide genes. These can also be referred to as “elimination genes”. A variety of suicide genes can be employed for this purpose, including HSV-TK (herpes simplex virus thymidine kinase), Fas, iCasp9 (inducible caspase 9), CD20, MYC TAG, and truncated EGFR (endothelial growth factor receptor). HSK for example, will convert the prodrug ganciclovir (GCV) into GCV-triphosphate that incorporates itself into replicating DNA, ultimately leading to cell death. iCasp9 is a chimeric protein containing components of FK506-binding protein that binds the small molecule AP1903, leading to caspase 9 dimerization and apoptosis. Other suitable suicide genes will be appreciated by those of ordinary skill in the art. Suicide genes can function to provide a route to specifically remove modified cells from a subject.

As used herein, “Tollip gene” refers to any polynucleotide that encodes a Tollip (Toll-interacting protein) protein or variant thereof “Tollip gene” can include Mouse Tollip Coding DNA GENEBANK ACCESSION #NC_000073; Human Tollip Coding DNA GENEBANK ACCESSION #CR533477 and any other variant including, but not limited to, homologues and orthologues thereof.

As used herein, “Tollip protein” refers to any polypeptide that is encoded by a Tollip (Toll-interacting protein) gene. The Tollip protein can have a polypeptide sequence such as that identified “Tollip protein” can include Human Tollip Protein GENEBANK ACCESSION #CAG38508; Mouse Tollip Protein GENEBANK ACCESSION #CAB58121 and any other variant including, but not limited to, homologues and orthologues thereof.

As used herein, “gene silencing oligonucleotide” refers to any oligonucleotide that can alone or with other gene silencing oligonucleotides utilize a cell's endogenous mechanisms, molecules, proteins, enzymes, and/or other cell machinery or exogenous molecule, agent, protein, enzyme, and/or polynucleotide to cause a global or specific reduction or elimination in gene expression, RNA level(s), RNA translation, RNA transcription, that can lead to a reduction or effective loss of a protein expression and/or function of a non-coding RNA as compared to wild-type or a suitable control. This is synonymous with the phrase “gene knockdown” Reduction in gene expression, RNA level(s), RNA translation, RNA transcription, and/or protein expression can range from about 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, to 1% or less reduction. “Gene silencing oligonucleotides” include, but are not limited to, any antisense oligonucleotide, ribozyme, any oligonucleotide (single or double stranded) used to stimulate the RNA interference (RNAi) pathway in a cell (collectively RNAi oligonucleotides), small interfering RNA (siRNA), microRNA, short-hairpin RNA (shRNA), and gRNAs for CRISPR. Commercially available programs and tools are available to design the nucleotide sequence of gene silencing oligonucleotides for a desired gene, based on the gene sequence and other information available to one of ordinary skill in the art.

### Overview

Decades of extensive studies have provided compelling evidence supporting a role for the immune system during the complex dynamics of tumor initiation, progression and regression. Most notably, the roles of adaptive immune cells such as T cells are well appreciated through recognizing tumor-specific antigens and coordinating anti-tumor functions. Recent advances suggest that innate immune cells including dendritic cells, monocytes and neutrophils play vital roles in facilitating the anti-tumor functions of T cells, through affecting the expression and activities of immune check point genes such as PD-L1. Despite these exciting advancements, it still remains less understood with regard to the roles and mechanisms of innate immune cells during the modulation of tumor-immune environment.

Among tumor infiltrating innate immune cells, neutrophil is one of the major constituents. Solid tumor patients with poor prognosis tend to have expanded pools of tumor-associated neutrophils. Although the mechanisms are not well understood, neutrophils are known to exhibit complex and often opposing functions that either facilitate or prevent tumor initiation and growth. Differential expressions of neutrophil cell surface molecules (e.g. PD-L1) as well as secretory mediators may contribute to the opposing functions of neutrophils in either augmenting or suppressing adaptive T cell activation. However, molecular mechanisms underlying the differential activation of neutrophils are not known.

Tollip (Toll interacting protein) is an innate immunity signaling adaptor molecule expressed in myeloid cells. Initially recognized as an inhibitor for the TLR signaling pathway, recent studies suggest that Tollip may modulate cellular autophagy and other pathways in monocyte. Its role in modulating neutrophil function and tumor immune environment has not been studied.

With that said, described herein are modified neutrophils and populations thereof that can have reduced or eliminated Tollip gene expression and/or amount of Tollip protein as compared to a wild-type or suitable control neutrophil. Also described herein are methods of generating the modified neutrophils and populations thereof. Also described herein are methods of administering the modified neutrophils and/or populations thereof to a subject. In some embodiments, the subject can have or be suspected of having a cancer. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Tollip Deficient Neutrophils

Described herein are Tollip deficient neutrophils. In some embodiments, the Tollip deficient neutrophils are modified neutrophils that have reduced or eliminated Tollip gene and/or protein expression as compared to a wild-type or unmodified neutrophil or suitable control. In some aspects the Tollip deficient neutrophils can have a reduced amount of Tollip mRNA and/or amount of Tollip protein as compared to a wild-type or unmodified neutrophil or suitable control. Suitable techniques for detecting and measuring gene and protein expression and/or amount of mRNA or protein will be instantly appreciated by one of ordinary skill in the art and are within the spirit and scope of this disclosure. Such techniques include but are not limited to, various PCR methods (e.g. RT-PCR, qPCR, RT-qPCR, ELISA, Western blotting, Northern blotting, nucleotide sequencing methods, and the like).

In some embodiments, amount of Tollip gene expression, amount of Tollip mRNA, and/or amount of Tollip protein in the modified neutrophils can be reduced by about 1, to about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 80, 81, 82, 83, 84, 85, 86, 87, 88, 89, 90, 91, 92, 93, 94, 95, 96, 97, 98, 99, or 100 percent. In some embodiments, amount of Tollip gene expression, amount of Tollip mRNA, and/or amount of Tollip protein in the modified neutrophils can be reduced by about 1, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 80, 81, 82, 83, 84, 85, 86, 87, 88, 89, 90, 91, 92, 93, 94, 95, 96, 97, 98, 99, or 100 percent.

The modified Tollip deficient neutrophil can have increased gene and/or protein expression of CD80 as compared to a wild-type neutrophil or suitable control. CD80 can include Human CD80 coding DNA (Genbank accession #NM_005191) and any variant thereof and Human CD80 protein (Genbank accession #AAH42665) and any variant thereof. The modified Tollip deficient neutrophil. The modified neutrophil can have decreased gene and/or protein expression of PDL-1 as compared to a wild-type neutrophil or a suitable control. PDL-1 can include Human PD-L1 gene coding DNA (Genbank accession #NM_014143) and any variant thereof and Human PD-L1 protein (Genbank accession #NP_054862) and any variant thereof.

In some embodiments, a modified neutrophil can include a deletion of one or more copies of the Tollip gene. In some embodiments, a modified neutrophil can include a Tollip gene silencing oligonucleotide and/or a Tollip gene silencing oligonucleotide expression vector.

The modified Tollip deficient neutrophils described herein can contain additional modifications. The modified Tollip deficient neutrophils can include one or more additional marker genes and/or molecular barcodes to allow for identification of the modified neutrophils. Suitable markers can include, but are not limited to, antibiotic resistance markers, fluorescent protein markers, enzyme markers, and cell surface markers. It will be appreciated that in some embodiments, the marker can be unique to the modified cells to allow them to be distinguishable from unmodified cells. In some embodiments, the markers can be a cell surface marker that can be bound by an antibody, which can allow for selective removal of modified neutrophils once administered. The modified Tollip deficient neutrophils described herein can include one or more suicide genes, which can allow for on-demand destruction of modified Tollip deficient neutrophils. Any of the additional modifications can be conditionally or constitutively expressed.

Also described herein are formulations, such as pharmaceutical formulations, that can include a modified Tollip deficient neutrophil as described herein or a population thereof and a carrier, such as a pharmaceutically acceptable carrier. The formulation, such as a pharmaceutical formulation, can include a therapeutically effective amount of the modified neutrophil or population thereof. The therapeutically effective amount can range from about 100 to 1×10₁, 1×10₂, 1×10₃, 1×10₄, 1×10₅, 1×10₆, 1×10₇, 1×10₈, 1×10₉, 1×10₁₀, 1×10₁₁, 1×10₁₂, 1×10₁₃, 1×10₁₄, 1×10₁₅, 1×10₁₆, 1×10₁₇, 1×10₁₈, 1×10₁₉, 1×10₂₀ or more cells/mL. The pharmaceutical formulations can be used to treat and/or prevent cancer and/or symptom thereof.

Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

In addition to the therapeutically effective amount of the Tollip deficient neutrophil or population thereof the pharmaceutical formulation can also include an effective amount of an auxiliary active agent, including but not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, chemotherapeutics and combinations thereof.

In embodiments where there is an auxiliary active agent contained in the pharmaceutical formulation in addition to the Tollip deficient neutrophil or population thereof, the therapeutically effective amount of the auxiliary active agent will vary depending on the auxiliary active agent. In some embodiments, the effective amount of the auxiliary active agent ranges from 0.001 micrograms to about 1 milligram, such as 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 microgams or milligrams. In other embodiments, the amount of the auxiliary active agent ranges from about 0.01 IU to about 1000 IU, such as about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or about 1000. In further embodiments, the amount of the auxiliary active agent ranges from 0.001 mL to about 1 mL, such as about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or about 1 mL. In yet other embodiments, the amount of the auxiliary active agent ranges from about 1% w/w to about 50% w/w, v/v, or w/v of the total pharmaceutical formulation, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, to/or 50% w/w, v/v, or w/v of the total pharmaceutical formulation.

### Dosage Forms

In some embodiments, the pharmaceutical formulations described herein may be in a dosage form. The dosage forms can be adapted for administration by any appropriate route. Appropriate routes can include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

Dosage forms adapted for parenteral administration and/or adapted for any type of injection (e.g. intravenous, intraperitoneal, subcutaneous, intramuscular, intradermal, intraosseous, epidural, intracardiac, intraarticular, intracavernous, gingival, subginigival, intrathecal, intravireal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from concentrated cell solutions, sterile powders, granules, and tablets.

For some embodiments, the dosage form contains a predetermined amount of the Tollip deficient neutrophils per unit dose. In some embodiments, the predetermined amount of the Tollip deficient neutrophils is a therapeutically effective amount of the Tollip deficient neutrophils effective to treat or prevent cancer or a symptom thereof. In other embodiments, the predetermined amount of the Tollip deficient neutrophils can be an appropriate fraction of the therapeutically effective amount of the active ingredient (e.g. the Tollip deficient neutrophils and/or auxiliary active agent). Such unit doses may therefore be administered once or more than once a day. Such pharmaceutical formulations may be prepared by any of the methods well known in the art.

### Methods of Generating Tollip Deficient Neutrophils

Also described herein are methods of generating a Tollip deficient neutrophil or population thereof. Chemical, biological, and/or genetic approaches to modify/reduce the levels/activities of Tollip in neutrophils may include siRNA (small interfering RNA against Tollip), CRISPR-CAS knockdown of Tollip expression, or incubation of neutrophils with very-low dose of endotoxin LPS (lipopolysaccharide) and/or its mimetics/derivatives as well as other compounds with similar effect of reducing the levels and/or activities of Tollip inside neutrophils or innate monocytes. Any suitable gene deletion, gene silencing techniques, and/or gene editing techniques can be used to generate the tollip deficient neutrophils described herein. It will be appreciated that as used herein gene deletion refers to techniques resulting in deletion of one or more copies of a gene. Thus, deleting the Tollip gene means that one or more of the Tollip gene copies have been deleted at the genomic level. Gene deletion is used synonymously with “gene knockout”. It will be appreciated that as used herein gene silencing is used to refer to techniques that do not necessarily remove one or more genomic copies of the gene, such as the Tollip gene, but rather can result an effective reduction of gene expression by altering the transcription of the gene (e.g. Tollip gene), mRNA levels (e.g. Tollip mRNA levels), translation of mRNA (e.g. translation of mRNA levels), which in turn can result in decreased protein expression (e.g. decreased Tollip protein expression). Gene editing techniques can be used to obtain effective gene deletions and gene knockdowns depending on how the genes are edited. For example, a gene can be edited to produce a mRNA transcript with lower stability, which may result in effective gene knockdown. In other cases, the gene (e.g. the Tollip gene) can be modified (by addition or deletion of nucleotides) such that the mRNA transcript no longer produces a functional Tollip protein because it is not translated, does not undergo proper post-translational processing, and/or forms a modified Tollip protein that is not functional. This would be an example of an effective gene knockout or deletion.

Suitable techniques for producing gene deletions, effective gene deletions, gene knockdowns, and effective gene knockdowns include, but are not limited to, various techniques that rely on homologous recombination, site-specific nuclease-based techniques (e.g. zinc-finger based techniques, TALEN (transcription activator-like effector nuclease)-based techniques, and CRISPR (clustered regularly interspaced short palindromic repeats), and knock in-based techniques. In some embodiments, an exogenous molecule or gene can be introduced that be or express a gene silencing oligonucleotide. The gene silencing oligonucleotide can then reduce the amount of a specific mRNA (e.g. a Tollip mRNA) and therefore result in a reduction or effective elimination of the Tollip protein. It will be appreciated that some techniques can be performed in vivo. It will be appreciated that some techniques can be performed ex vivo or in vitro.

In some embodiments, Tollip deficient neutrophils can be generated by harvesting neutrophils from a subject to obtain harvested neutrophils and deleting or effectively deleting one or more copies of the Tollip gene in one or more of the harvested neutrophils in vitro to obtain the Tollip deficient neutrophil or population thereof. The Tollip deficient neutrophil or population thereof can have reduced, effectively eliminated, or eliminated Tollip gene expression and/or amount of Tollip protein as compared to a wild-type or suitable control neutrophil. The Tollip deficient neutrophil or population thereof can have increased gene and/or protein expression of CD80 as compared to a wild-type neutrophil or suitable control. The Tollip deficient neutrophil or population thereof can have decreased gene and/or protein expression of PDL-las compared to a wild-type neutrophil or suitable control. In some embodiments, the method can further include the step of transforming a harvested neutrophil or the Tollip deficient neutrophil or population thereof to contain and/or conditionally express a suicide gene. In some embodiments, the subject from which the neutrophils are harvested can be a human or another mammal.

In some embodiments, the method of generating a Tollip deficient neutrophil or population thereof can include the step of transforming a neutrophil with a Tollip gene silencing oligonucleotide or gene silencing oligonucleotide expression vector or gene to generate the Tollip deficient neutrophil or population thereof. The step of transforming can be performed in vitro, ex vivo, or in vivo. The method can further include the step of transforming a neutrophil with a suicide gene. The method can further include the step of harvesting neutrophils from a subject to obtain harvested neutrophils and wherein the one or more of the harvested neutrophils are transformed in vitro to generate a Tollip deficient neutrophil or population thereof.

In some embodiments, Tollip expression can be decreased and/or eliminated by exposing a neutrophil to a low amount of an endotoxin, including, but not limited to, LPS. In some embodiments, the low amount of endotoxin, such as LPS, can range from about 1-1000 pg/mL, such as about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or/to 1000 pg/mL. In some embodiments, the low amount of endotoxin, such as LPS, can range from about 0.1-10 EU/mL, such as about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, to/or 10 EU/mL.

The methods can include various culturing steps to maintain, grow, expand, and store the harvested and/or modified Tollip deficient neutrophils described herein. The Tollip deficient neutrophils can be cultured, grown, expanded, stored, harvested and/or otherwise modified prior to administration to a subject in need thereof. Such techniques will be instantly appreciated by those of ordinary skill in the art and are within the spirit and scope of this disclosure.

### Methods of Using the Tollip Deficient Neutrophils

Described herein are methods of using the Tollip deficient neutrophils described herein. In some embodiments, a Tollip deficient neutrophil or population thereof and/or Tollip gene silencing oligonucleotide can be administered to a subject. In some embodiments, the subject can be a subject in need thereof. In some aspects the subject can have or be suspected of having a cancer and/or a symptom thereof. In some embodiments, the Tollip deficient neutrophil or population thereof can be essentially autologous. In other words, the neutrophils that were modified ex vivo were harvested from the same subject they are administered to after modification. In some embodiments, the Tollip deficient neutrophil can be autologous. In other words, the neutrophils that were modified ex vivo were harvested from a different subject than they are administered to after modification.

As described elsewhere herein, the neutrophils can be transformed in vivo. Thus, in some embodiments, a gene silencing oligonucleotide and/or a gene silencing oligonucleotide can be administered to a subject in need thereof. The subject in need thereof can have or be suspected of having a cancer. In some aspects, the gene silencing oligonucleotide and/or expression vector can enter a neutrophil, transform the neutrophil, and/or work to reduce or effectively eliminate Tollip gene expression in the neutrophil. Additional markers and/or suicide genes or expression vectors can be delivered along with the gene silencing oligonucleotide to allow for identification, selection, and/or removal of transformed neutrophils.

Also described herein are methods of treating and/or preventing a cancer or a symptom thereof in a subject that can include the step of administering a Tollip deficient neutrophil or population thereof and/or Tollip gene silencing oligonucleotide as described herein and/or gene silencing oligonucleotide expression vector to the subject.

An amount of the Tollip deficient neutrophil or population thereof and/or Tollip gene silencing oligonucleotide and/or pharmaceutical formulations thereof described herein can be administered to a subject in need thereof one or more times per day, week, month, or year. In some embodiments, the amount administered can be the therapeutically effective amount of the Tollip deficient neutrophil or population thereof and/or Tollip gene silencing oligonucleotide and/or pharmaceutical formulations thereof described herein or pharmaceutical formulations thereof. For example, the Tollip deficient neutrophil or population thereof and/or Tollip gene silencing oligonucleotide and/or pharmaceutical formulations thereof described herein can be administered in a daily dose. This amount may be given in a single dose per day. In other embodiments, the daily dose may be administered over multiple doses per day, in which each containing a fraction of the total daily dose to be administered (sub-doses). In some embodiments, the number of doses delivered per day is 2, 3, 4, 5, or 6. In further embodiments, the Tollip deficient neutrophil or population thereof and/or Tollip gene silencing oligonucleotide and/or pharmaceutical formulations thereof described herein can be administered one or more times per week, such as 1, 2, 3, 4, 5, or 6 times per week. In other embodiments, the Tollip deficient neutrophil or population thereof and/or Tollip gene silencing oligonucleotide and/or pharmaceutical formulations thereof described herein can be administered one or more times per month, such as 1 to 5 or more, such as 1, 2, 3, 4, 5 or more times per month. In still further embodiments, the Tollip deficient neutrophil or population thereof and/or Tollip gene silencing oligonucleotide and/or pharmaceutical formulations thereof described herein can be administered one or more times per year, such as 1 to 11 or more (e.g. 1, 2, 3, 4, 5, 6, 7, 8, 9, 10, 11, or more) times per year.

The Tollip deficient neutrophil or population thereof and/or Tollip gene silencing oligonucleotide and/or pharmaceutical formulations thereof described herein can be co-administered with a secondary agent by any convenient route. The secondary agent is a separate compound and/or pharmaceutical formulation from Tollip deficient neutrophil or population thereof and/or Tollip gene silencing oligonucleotide and/or pharmaceutical formulations thereof described herein. The secondary agent can be administered simultaneously with the Tollip deficient neutrophil or population thereof and/or Tollip gene silencing oligonucleotide and/or pharmaceutical formulations thereof described herein. The secondary agent can be administered sequentially with the Tollip deficient neutrophil or population thereof and/or Tollip gene silencing oligonucleotide and/or pharmaceutical formulations thereof described herein. Suitable secondary agents include, but are not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics.

In embodiments where the Tollip deficient neutrophil or population thereof and/or Tollip gene silencing oligonucleotide and/or pharmaceutical formulations thereof described herein are simultaneously co-administered with a secondary agent, the Tollip deficient neutrophil or population thereof and/or Tollip gene silencing oligonucleotide and/or pharmaceutical formulations thereof described herein can be administered to the subject at substantially the same time as the secondary agent. As used in this context “substantially the same time” refers to administration of Tollip deficient neutrophil or population thereof and/or Tollip gene silencing oligonucleotide and/or pharmaceutical formulations thereof described herein and a secondary agent where the period of time between administration of the Tollip deficient neutrophil or population thereof and/or Tollip gene silencing oligonucleotide and/or pharmaceutical formulations thereof described herein and the secondary agent is between 0 and 10 minutes, such as 0, 1, 2, 3, 4, 5, 6, 7, 8, 9, or 10 minutes.

In embodiments where the Tollip deficient neutrophil or population thereof and/or Tollip gene silencing oligonucleotide and/or pharmaceutical formulations thereof described herein is/are sequentially co-administered with a secondary agent, the Tollip deficient neutrophil or population thereof and/or Tollip gene silencing oligonucleotide and/or pharmaceutical formulations thereof described herein can be administered first, and followed by administration of the secondary agent after a period of time. In other embodiments where the Tollip deficient neutrophil or population thereof and/or Tollip gene silencing oligonucleotide and/or pharmaceutical formulations thereof described herein is/are sequentially co-administered with a secondary agent, the secondary agent can be administered first, and followed by administration of the Tollip deficient neutrophil or population thereof and/or Tollip gene silencing oligonucleotide and/or pharmaceutical formulations thereof described herein after a period of time. The period of time between administration of the Tollip deficient neutrophil or population thereof and/or Tollip gene silencing oligonucleotide and/or pharmaceutical formulations thereof described herein and the secondary agent can range from 10 minutes to about 96 hours. In some embodiments, the period of time can be about 10 minutes, about 30 minutes, about 1 hour, about 2 hours, about 4 hours, about 6 hours, about 8 hours, about 10 hours, or about 12 hours. In some embodiments, the period of time can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 min. In some embodiments, the period of time can eb about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours. The sequential administration can be repeated as necessary over the course of the period of treatment.

The amount of the Tollip deficient neutrophil or population thereof and/or Tollip gene silencing oligonucleotide and/or pharmaceutical formulations thereof described herein that can be administered are described elsewhere herein. The amount of the secondary agent will vary depending on the secondary agent. The amount of the secondary agent can be a therapeutically effective amount. In some embodiments, the effective amount of the secondary agent ranges from 0.001 micrograms to about 1 milligram, such as 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 microgams or milligrams. In other embodiments, the amount of the secondary agent ranges from about 0.01 IU to about 1000 IU, such as about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or about 1000 IU. In further embodiments, the amount of the secondary agent ranges from 0.001 mL to about 1 mL, such as about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or about 1 mL. In yet other embodiments, the amount of the secondary agent ranges from about 1% w/w to about 50% w/w, v/v, or w/v of the total pharmaceutical formulation, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, to/or 50% w/w, v/v, or w/v of the total pharmaceutical formulation.

In some embodiments, the Tollip deficient neutrophil or population thereof and/or Tollip gene silencing oligonucleotide and/or pharmaceutical formulations thereof described herein can be administered to a patient via an injection. Suitable methods of injection include, but are not limited to, intravenous, intraperitoneal, subcutaneous, intramuscular, intradermal, intraosseous, epidural, intracardiac, intraarticular, intracavernous, intrathecal, intravitreal, intracerebral, gingival, subginigival, intranodal, and intracerebroventricular injection. Other suitable methods of administration of the composition or formulation containing the Tollip deficient neutrophil or population thereof and/or Tollip gene silencing oligonucleotide and/or pharmaceutical formulations thereof can include, but are not limited to, subcutaneous, intravenous, parenteral, and/or oral delivery. In some embodiments, the dosage of the Tollip deficient neutrophil or population thereof and/or Tollip gene silencing oligonucleotide and/or pharmaceutical formulations thereof can range from about 0.01 μg/kg bodyweight to about 1 mg/kg bodyweight.

Kits containing the Tollip Deficient Neutrophils and/or Tollip Gene Silencing Oligonucleotide and/or Pharmaceutical Formulations Thereof

The Tollip deficient neutrophil or population thereof and/or Tollip gene silencing oligonucleotide and/or pharmaceutical formulations thereof can be presented as a combination kit. As used herein, the terms “combination kit” or “kit of parts” refers to the chemically programmed neutrophils or pharmaceutical formulations thereof and compositions and pharmaceutical formulations thereof described herein and additional components that are used to package, sell, market, deliver, and/or administer the combination of elements or a single element, such as the active ingredient, contained therein. Such additional components include but are not limited to, packaging, syringes, blister packages, bottles, and the like. When one or more of the components (e.g. active agents) contained in the kit are administered simultaneously, the combination kit can contain the active agents in a single pharmaceutical formulation (e.g. a tablet) or in separate pharmaceutical formulations.

The combination kit can contain each agent, compound, pharmaceutical formulation or component thereof described herein, in separate compositions or pharmaceutical formulations. The separate compositions or pharmaceutical formulations can be contained in a single package or in separate packages within the kit. Also provided in some embodiments, are buffers, diluents, solubilization reagents, cell culture media and other reagents. These additional components can be contained in a single package or in separate packages within the kit.

In some embodiments, the combination kit also includes instructions printed on or otherwise contained in a tangible medium of expression. The instructions can provide information regarding the content of the Tollip deficient neutrophil or population thereof and/or Tollip gene silencing oligonucleotide and/or pharmaceutical formulations thereof and/or other auxiliary and/or secondary agent contained therein, safety information regarding the safety of the Tollip deficient neutrophil or population thereof and/or Tollip gene silencing oligonucleotide and/or pharmaceutical formulations thereof and/or other auxiliary and/or secondary agent contained therein, information regarding the dosages, indications for use, and/or recommended treatment regimen(s) for the Tollip deficient neutrophil or population thereof and/or Tollip gene silencing oligonucleotide and/or pharmaceutical formulations thereof and/or other auxiliary and/or secondary agent contained therein. In some embodiments, the instructions can provide directions for administering the Tollip deficient neutrophil or population thereof and/or Tollip gene silencing oligonucleotide and/or pharmaceutical formulations thereof and/or other auxiliary and/or secondary agent to a subject having or suspected of cancer and/or a symptom thereof.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

Decades of extensive studies have provided compelling evidence supporting a role for the immune system during the complex dynamics of tumor initiation, progression and regression (1). Most notably, the roles of adaptive immune cells such as T cells are well appreciated through recognizing tumor-specific antigens and coordinating anti-tumor functions (2). Recent advances suggest that innate immune cells including dendritic cells, monocytes and neutrophils play vital roles in facilitating the anti-tumor functions of T cells, through affecting the expression and activities of immune check point genes such as PD-L1 (3). Despite these exciting advancements, it still remains less understood with regard to the roles and mechanisms of innate immune cells during the modulation of tumor-immune environment.

Among tumor infiltrating innate immune cells, neutrophil is one of the major constituents (4). Solid tumor patients with poor prognosis tend to have expanded pools of tumor-associated neutrophils (4-6). Although the mechanisms are not well understood, neutrophils are known to exhibit complex and often opposing functions that either facilitate or prevent tumor initiation and growth (7). Differential expressions of neutrophil cell surface molecules (e.g. PD-L1) as well as secretory mediators may contribute to the opposing functions of neutrophils in either augmenting or suppressing adaptive T cell activation. However, molecular mechanisms underlying the differential activation of neutrophils are not known.

Tollip is an innate immunity signaling adaptor molecule expressed in myeloid cells (8). Initially recognized as an inhibitor for the TLR signaling pathway, recent studies suggest that Tollip may modulate cellular autophagy and other pathways in monocytes (9, 10). Its role in modulating neutrophil function and tumor immune environment has not been studied.

This Example can demonstrate that Tollip can contribute to the differential activation of neutrophils and that Tollip deficient neutrophils may alter tumor immune environment. This Example can demonstrate the results of testing the tumor burden, immune environment, and neutrophil function of WT and Tollip deficient mice bearing colon tumors chemically induced by AOM-DSS. Adoptive transfer studies were also performed to specifically examine the role of Tollip deficient neutrophils in modulating immune environment and tumor growth. It was observed that Tollip deficient mice have reduced tumor burden and enhanced neutrophil function in promoting T cell proliferation. Tollip deficient neutrophils have elevated co-stimulatory molecule CD80 and reduced co-inhibitory molecular PD-L1, through the induction of STAT5 and reduction of STAT1. This Example can further demonstrate that Tollip can be a molecular check-point that governs the decision-making processes of neutrophils in modulating tumor immune environment.

### Materials and Methods

Mice. Wild type (WT) C57BL/6 mice and Tollip−/− mice were bred and maintained in the animal facility at Virginia Tech in accordance to approved Animal Care and Use Committee protocol. All littermate mice were 8-10 weeks of age and 25-30 g weight when experiments were initiated.

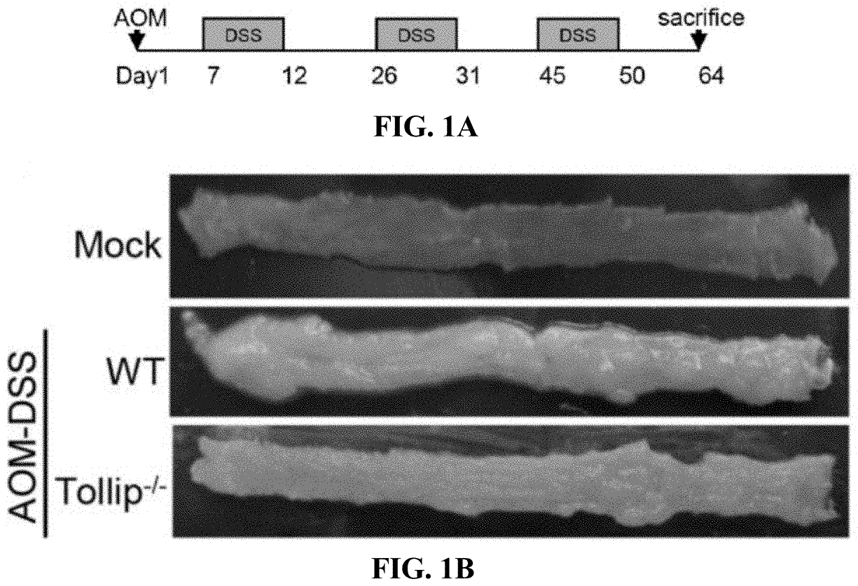

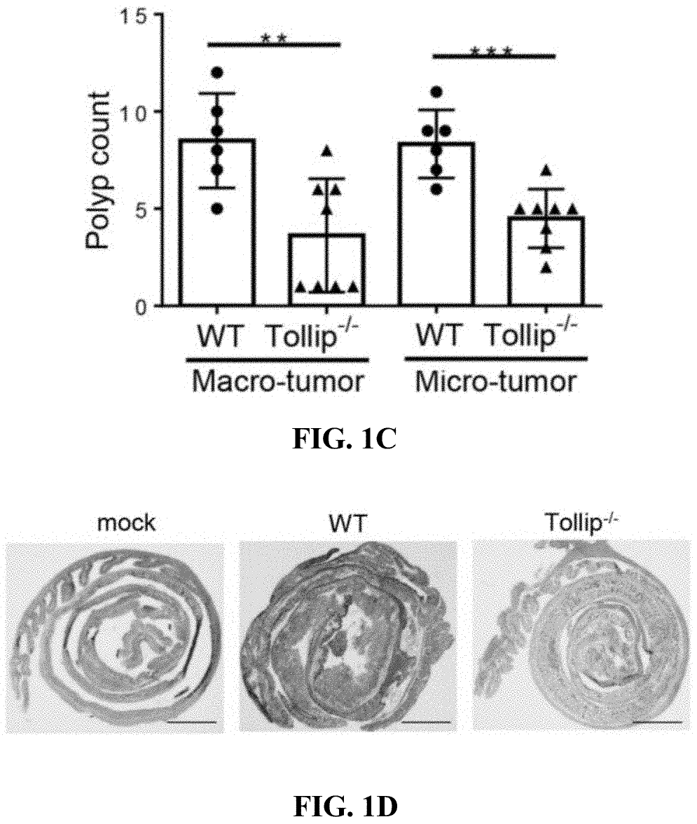

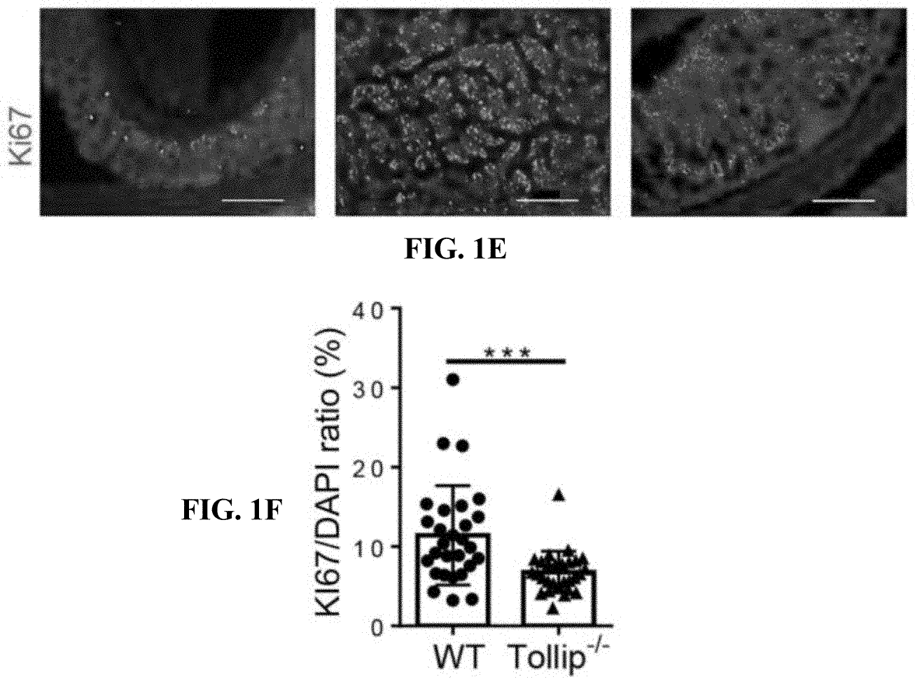

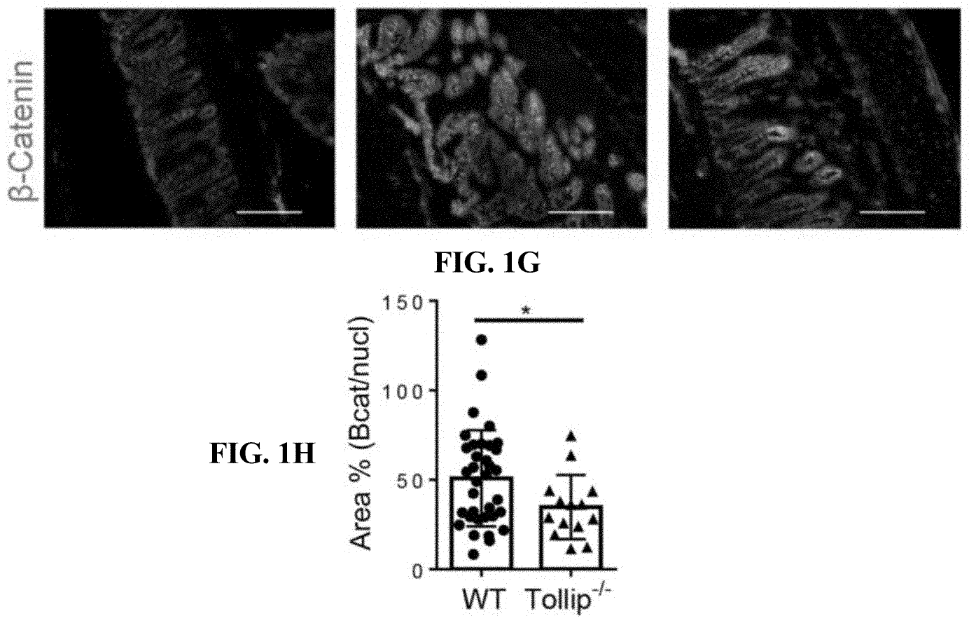

Experimental design. WT and Tollip−/− mice received a single intraperitoneal injection of azoxymethane (AOM, Sigma-Aldrich) at a dose of 10 mg/kg body weight. A week after AOM injection, the mice were given three cycles of 2% dextran sulfate sodium salt (DSS, MP Biomedicals) for 5 days followed by 14 days of normal drinking water. After the last water cycle mice were sacrificed and tissues were harvested for further analysis. A schematic protocol is illustrated in FIG. 1A. Body weight, stool consistency, bleeding was measured as part of clinical score (score 0-4, with higher score corresponding to worse condition). Polyp formation was labeled macro- and micro-polyp depending on the size equal to or greater than 2 mm versus less than 2 mm, respectively. Independent experiments of AOM-DSS induced colorectal tumorigenesis were conducted more than 3 times, and for every experiment there were at least 5 mice in each group.

Histology. Histological analyses of colon tissues were performed on freshly frozen OCT (Optimal-Cutting-Temperature compound)-embedded, and sectioned slides (5 μm). Slides were fixed in 4% neutral buffered formalin for 5 min. Haematoxylin and eosin (H&E) staining were performed.

Immunofluorescence. Immunofluorescence analyses were performed on freshly frozen OCT-embedded, and sectioned slides (5 μm). At least 6 mice from WT and Tollip−/− mice were used for the study. For the measurement of Ki67 (Abcam) and β-catenin (Cell Signaling), sections were fixed in 4% neutral buffered formalin for 5 min, and stained with anti-mouse primary antibodies (1:100) followed by a biotinylated anti-Ig secondary Ab (BD eBiosciences) and streptavidin-PE or FITC. DAPI was used to stain nucleus. Multiple viewing fields from each slide were captured under fluorescent microscope. Pixel values reflecting the fluorescent intensities of each viewing field were quantitated with the NIH ImageJ software.

Immunohistochemistry. Sections from WT and Tollip−/− were co-stained with anti-myeloperoxidase (MPO) (Abcam; 1:100 dilution) and anti-CD3 (Abcam; 1:100 dilution). Secondary staining was performed using VECTASTAIN Elite ABC-HRP Kit followed by DAB Peroxidase Substrate kit, or ImmPRESS™-AP Anti-Rat IgG Alkaline Phosphatase (AP) Polymer Detection Kit, followed by ImmPACT Vector Red AP Substrate. All kits were purchased from Vector laboratory. Staining was performed according to the manufacturer's instructions.

Immunoblotting. Bone marrow neutrophils were purified by 65% percoll gradient and the purity was >90% confirmed by Ly6G+CD11b+ staining. For neutrophil culture, purified neutrophils were cultured in RPMI 1640 medium supplemented with 10% fetal bovine serum, 2 mM L-glutamine, 10 mM HEPES, 1% penicillin/streptomycin and with GM-CSF (1 ng/ml) overnight. Naive or cultured neutrophils were harvested in SDS lysis buffer containing protease and phosphatase inhibitors as previously described (17). Briefly, equal amount of protein was applied to SDS-PAGE and transferred to PVDF membranes (BioRad). The membranes were blocked with 5% non-fat dry milk, and then incubated with primary anti-phospho STAT1 (cell signaling), anti-STAT1 (cell signaling), anti-phospho-p38 (Cell Signaling), anti-p38 (Cell Signaling), anti-phospho-p65 (Cell Signaling), anti-p65 (Cell Signaling), IRF5 (Cell Signaling), oxCaMKII (Millipore) or R-actin antibody (Santa Cruz), and anti-rabbit or mouse IgG secondary antibody (Cell Signaling) according to the manufacturer's instructions. The immunoblots were developed by a chemiluminescence ECL detection kit (Thermo Scientific).

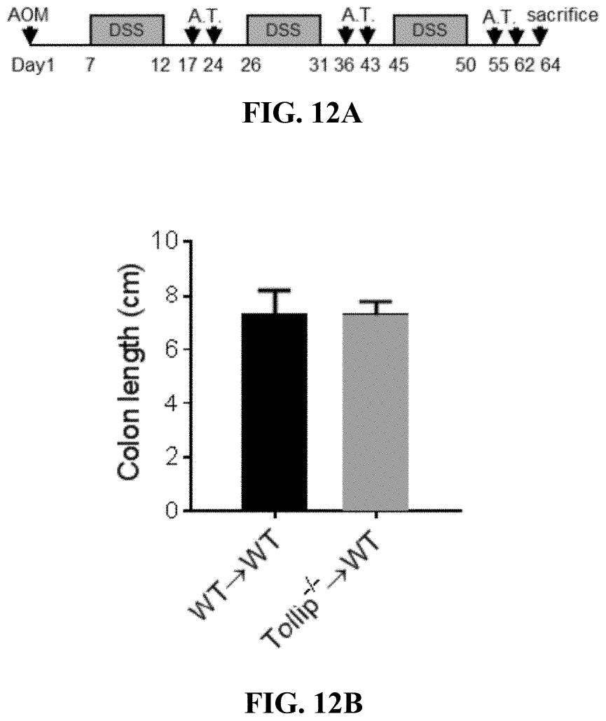

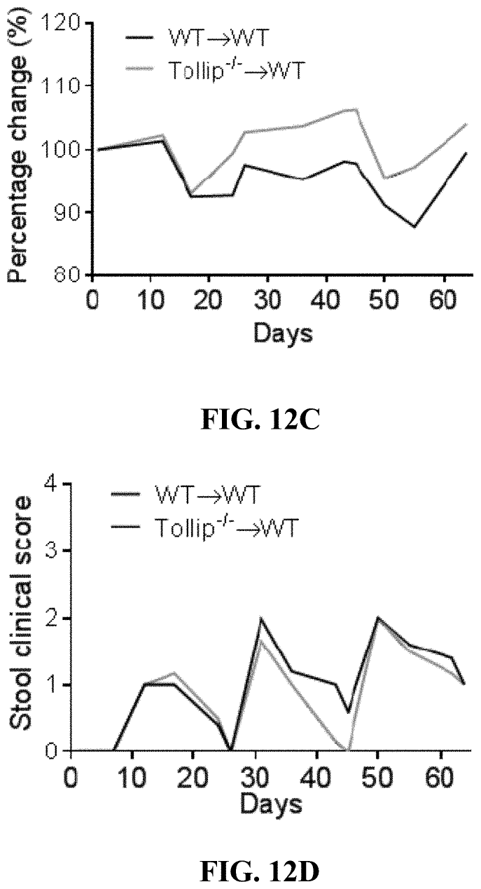

Adoptive transfer of neutrophils. Bone marrow neutrophils from donor mice (WT or Tollip−/−) were purified (>90% confirmed by flow cytometry) using EasySep™ Mouse Neutrophil Enrichment Kit (Stem Cell), according to the manufacturer's instruction. Recipient WT mice were transfused twice (post DSS day 5 and day 12) per DSS-resting cycle through intravenous injection with 2.4×106 WT or Tollip−/− neutrophils suspended in 200 μl sterile PBS. Detailed timeline was illustrated in FIGS. 12A-12D.

ELISA. The levels of TNF-α and LTB4 in plasma were measured using ELISA kits purchased from R&D system, according to the manufacturer's instructions.

Lamina propria cell isolation. Colons were opened longitudinally, and cleaned by flushing with ice-cold PBS. Single-cell suspension was prepared using Lamina Propria dissociation Kit (MACS). Briefly, the colons were cut into fine pieces, and incubated with HBSS containing 5 mM EDTA, 5% FBS, and 1 mM DTT to remove epithelial cells. The remaining pieces were then incubated with HBSS containing 5% FBS and enzyme mix using the gentleMACS dissociator. The cells were washed, passed through 70 μm strainer, and resuspended in FACS buffer for further flow cytometry analyses.

Flow cytometry. Fluorescent-conjugated anti-mouse antibodies specific for PD-L1, CD80, CD14, CD11 b, CD4, CD8, Ly6G were purchased from BioLegend. Propidium iodide (PI) was also added to determine the cell viability. Peripheral blood cells and splenocytes were harvested from WT and Tollip−/− mice as previously described (18). The cells were washed in FACS buffer (HBSS supplemented with 2% FBS and 0.02% sodium azide) and stained with fluorescently-labelled antibodies for 20 min on ice. Stained cells were analyzed with a FACSCanto II (BD Biosciences). FACS plots shown were analyzed with FlowJo (Ashland, Oreg.).

T cell proliferation assay. Splenocytes were labelled with 5,6-carboxyfluorescein diacetate succinimidyl (CFSE) (Invitrogen, Molecular Probe), according to the manufacturer's instructions. CFSE-labeled splenocytes were stimulated with plate-bound anti-mouse CD3 antibody (eBioscience, clone: xxx). Neutrophils purified from bone marrow were cultured in RPMI 1640 medium supplemented with 10% fetal bovine serum, 2 mM L-glutamine, 10 mM HEPES, 1% penicillin/streptomycin and with GM-CSF (1 ng/ml) for 24 hours. CFSE-labeled splenocytes were mixed with cultured neutrophils at a 1:1 ratio and co-cultured in CD3 coated plates for 72 hours. CFSE signals were analyzed by flow cytometry on gated CD4+ and CD8+ cells. In blocking experiments, antibodies against CD80 or PD-L1 (BioLegend) were add to the co-culture at the concentration of 1 ug/ml. To test the viability of T cells after co-culture, neutrophils and T cells were treated as above but plated in 96-well U bottom plate without CD3 coating.

Statistical analysis. All experiments were performed at least for 3 times. Representative and reproducible results were shown. Statistical analysis was performed with Prism software (GraphPad Software 6.0). Values were expressed as means±SD. The significance of the differences was assessed by Student's t-test. P<0.05 was considered statistically significant.

### Results

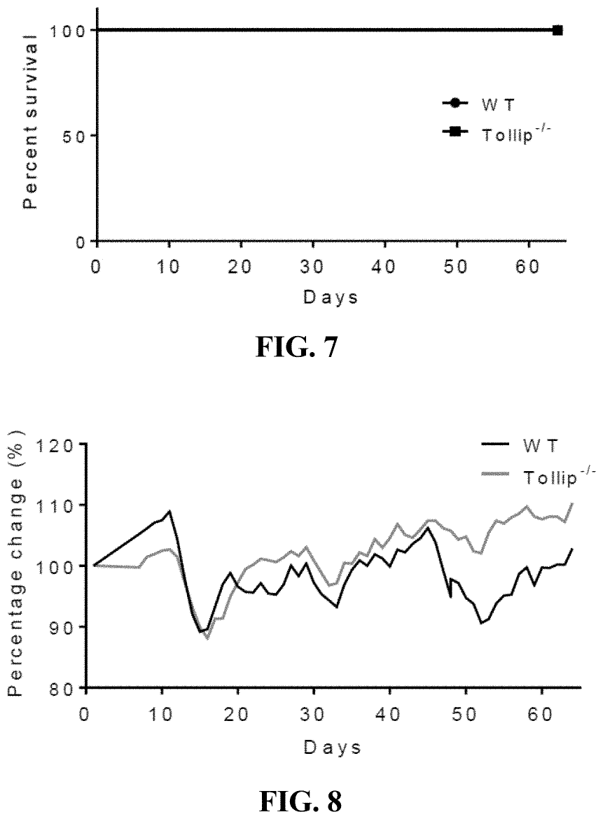

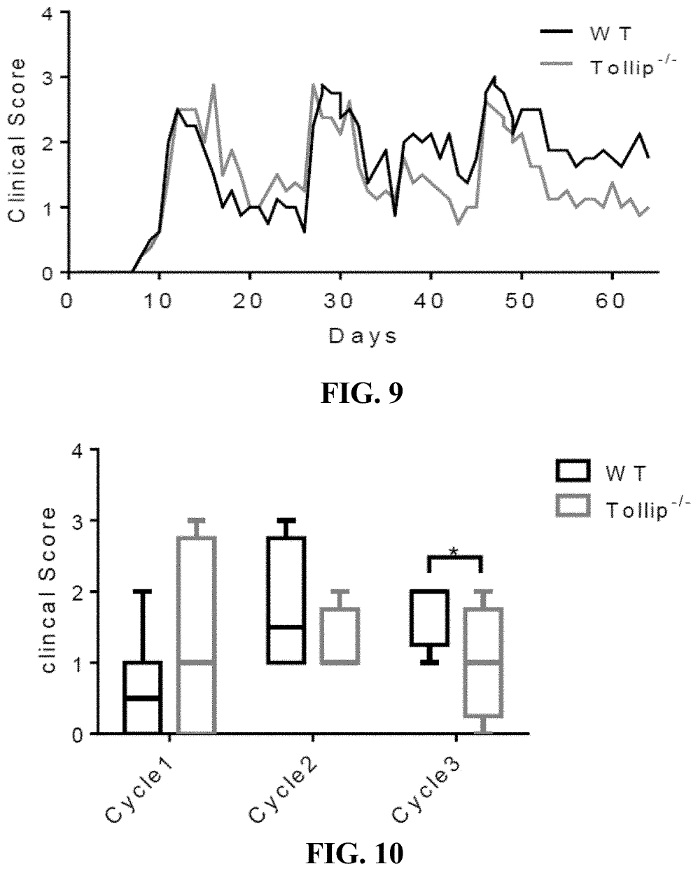

Tollip deficiency reduces colitis-associated tumorigenesis. In a previous study of acute DSS-induced colitis, it was observed that Tollip−/− mice exhibit more severe acute colitis as compared to WT mice, due to elevated leukocyte infiltration and inflammation in the gut tissue (11). In this Example, the severity of colorectal tumorigenesis in Tollip−/− mice was tested with the Azoxymethane (AOM)-dextran sulfate sodium salt (DSS) model, which is a well-defined colorectal cancer model. Age and gender compatible mice (WT and Tollip−/−) were injected i.p. with a single dose of AOM (10 mg/kg) followed by three cycles of 2% DSS in drinking water. While all mice survived the experimental periods (FIG. 7), WT mice developed significant amount of colon tumors throughout the distal as well as other segments of the colons (FIGS. 1B and 1C). In contrast, Tollip−/− mice exhibited 50% reduction in both microscopic and macroscopic polyps as compared to WT mice (FIGS. 1B and 1C). The whole body health conditions including weight loss, stool consistency, colorectal bleeding throughout the experimental course was monitored. Consistent with the acute colitis observations, observed slightly severe disease scores from Tollip−/− mice following the initial cycle of DSS were observed as compared to WT mice. In contrast, toward the end of the final DSS cycle, WT mice had much worse clinical scores including stool bleeding, consistent with more severe tumor burdens (FIGS. 8-10).

In addition to the general body outlook, histological assessment of the colon tissues was also performed. H&E staining showed more severe colon inflammation and alterations of epithelial structure in WT mice as compared to Tollip−/− mice (FIG. 1D). In addition, WT mice with the AOM-DSS treatment exhibited pervasive Ki67 staining throughout the colon tissues which served as a molecular marker for hyper-proliferative cells and tumorigenesis (FIG. 1E). In contrast, Tollip−/− mice similarly challenged with AOM-DSS demonstrated significantly reduced Ki67 positive cells in the colon (FIGS. 1E and 1F). Furthermore, the cellular levels of β-catenin were also decreased in the colon sections from Tollip−/− mice as compared to WT mice (FIGS. 1G and 1H). Collectively, these data reveal that Tollip deficient mice have reduced colon tumor formation when challenged with AOM-DSS.

### Tollip Deficiency Enhances Anti-Tumor Innate Immune Checkpoints and Facilitates Inflammation Homeostasis.

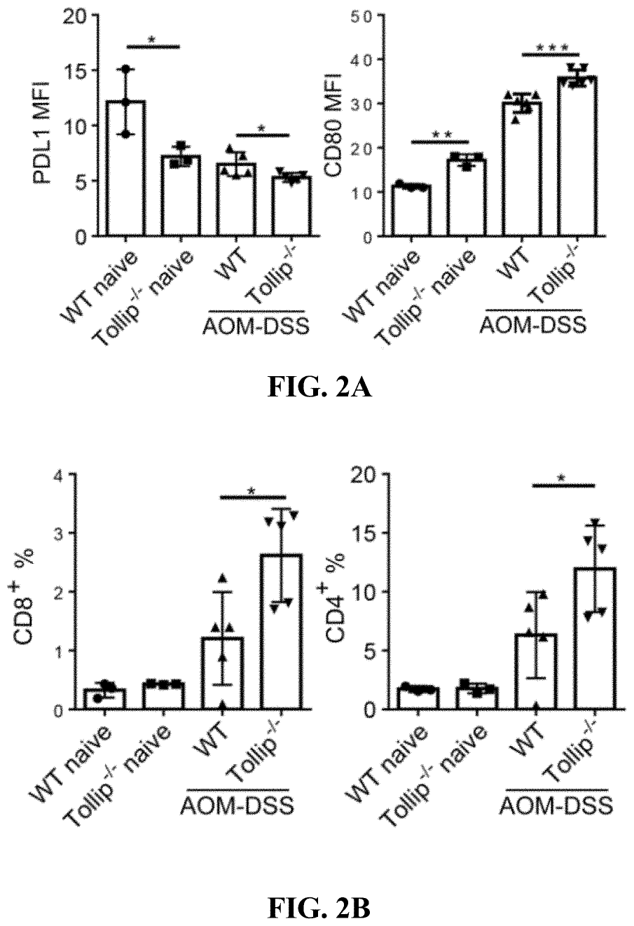

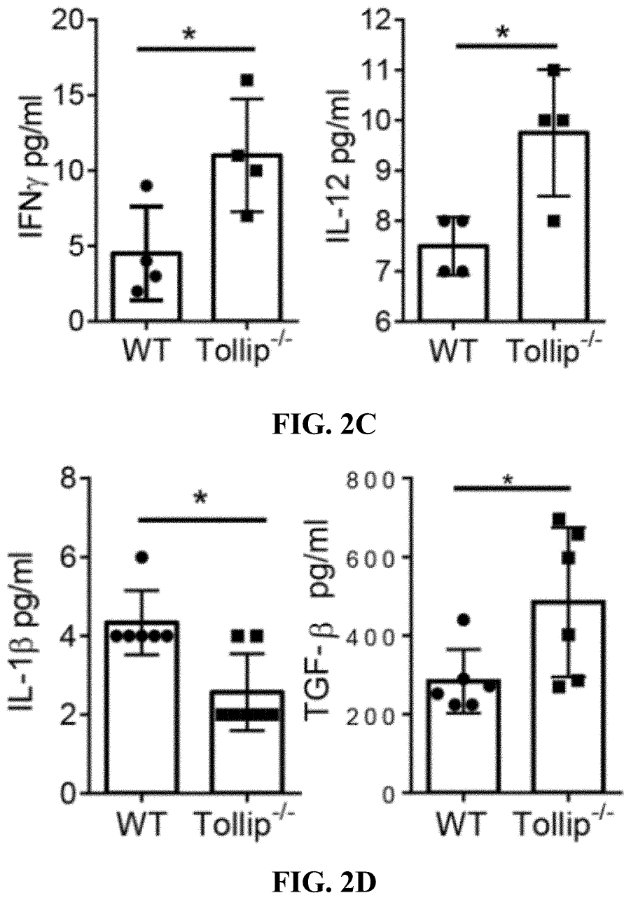

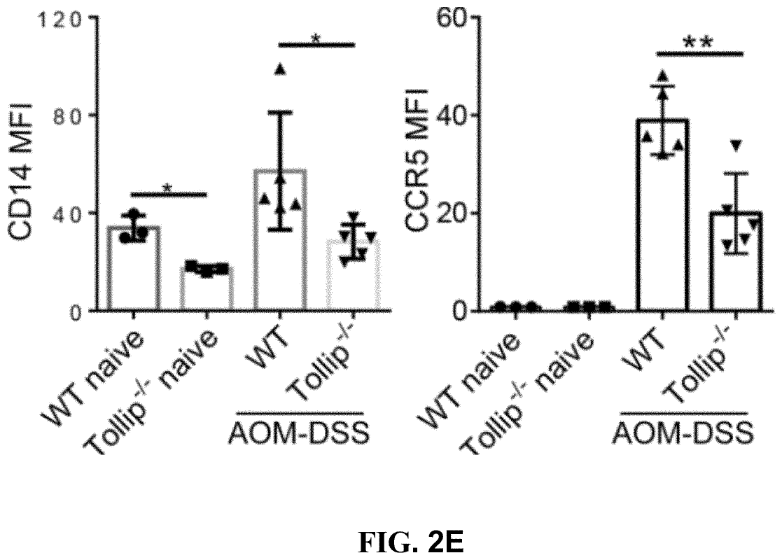

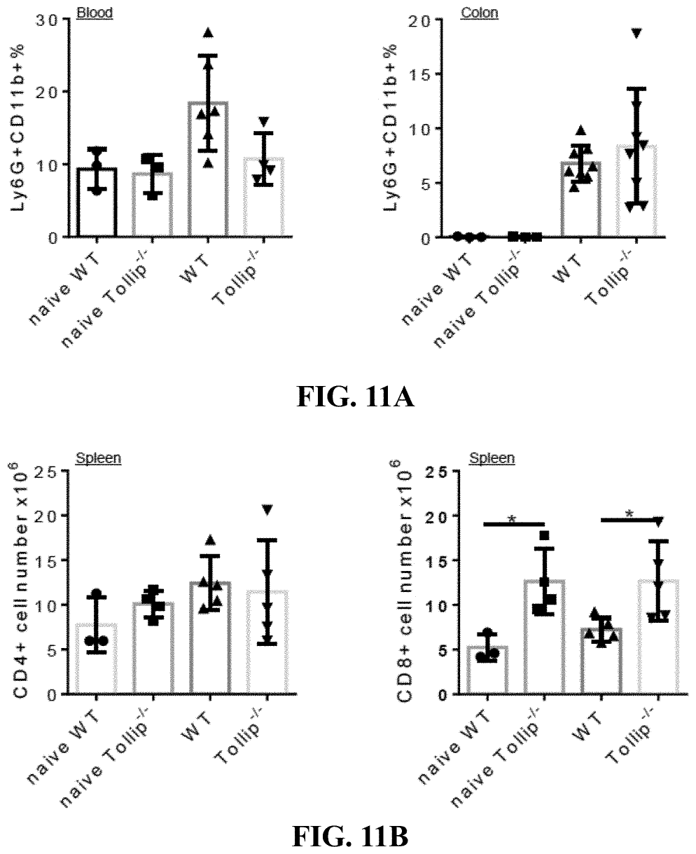

Since Tollip is recognized as a key modulator of innate immune cells, it was examined whether enhanced anti-tumor defense in Tollip deficient mice may be due to more effective anti-cancer checkpoints from innate immune cells. To test this, the key innate checkpoint molecules such as PD-L1 and CD80 expressed on neutrophils was measured. As shown in FIG. 2A, splenic neutrophils from naïve Tollip−/− mice expressed significantly less PD-L1 and higher CD80 as compared to naïve WT mice. This trend remained at the end of AOM-DSS cycle (FIG. 2A). The percentages of neutrophils within blood and colon tissues were similar among WT and Tollip mice before and after AOM-DSS challenge (FIGS. 11A-11B).

Next, CD4 T and CD8 T cells in WT and Tollip−/− mice was examined. At the end of the final DSS cycle, Tollip−/− mice had significantly higher amount of both CD4 and CD8 T cells as compared to WT mice within the lamina propria where colonic leukocytes home into (FIG. 2B). The numbers of CD8 T cells in the spleen were also significantly higher from Tollip−/− mice as compared to WT mice (FIGS. 11A-11B). Correlated with elevated T cell populations, weobserved elevated levels of IFNγ and IL-12 within colon tissues of Tollip−/− mice as compared to WT mice following AOM-DSS challenge (FIG. 2C).

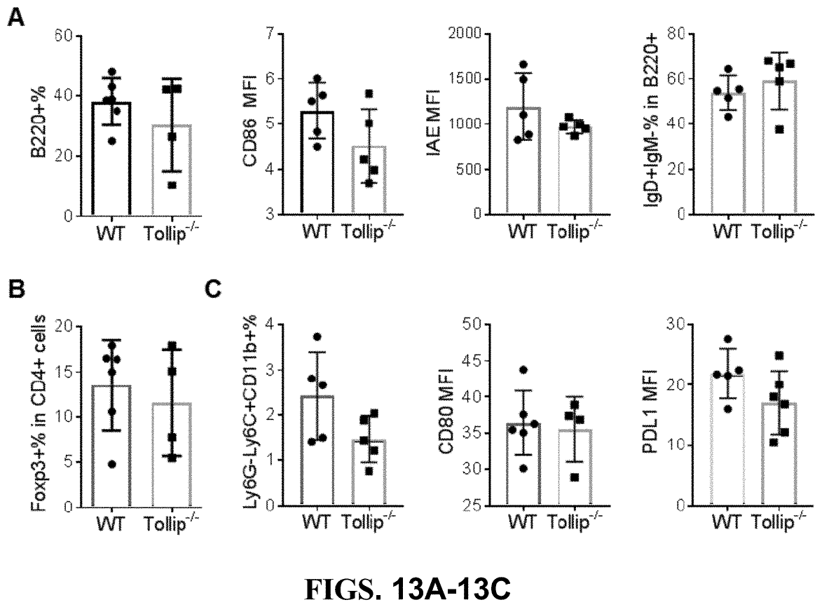

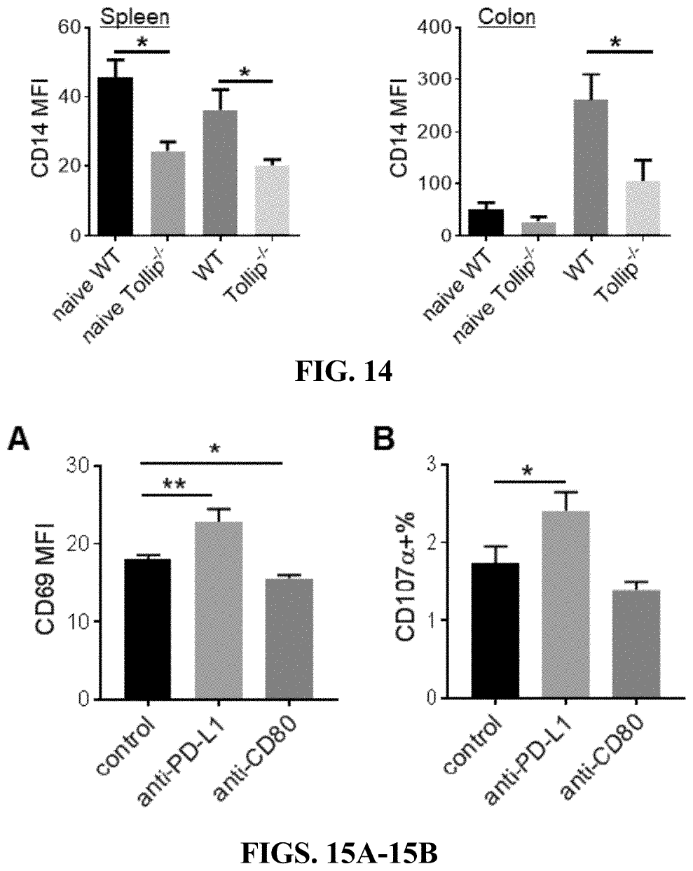

Despite elevated T cells and enhanced T-cell promoting neutrophils in Tollip−/− mice, a reduced circulating inflammatory cytokine IL-1β in Tollip−/− mice challenged with AOM-DSS as compared to WT mice was observed (FIG. 2D). Circulating plasma levels of TGF-β were significantly higher in Tollip−/− mice challenged with AOM-DSS as compared to WT mice (FIG. 2D). Other inflammatory surface markers of circulating neutrophils such as CD14 and CCR5 were also significantly lower in Tollip−/− mice as compared to WT mice (FIG. 2E). We also observed a similar reduction of CD14 on neutrophils collected from spleen and colon tissues of Tollip-deficient mice as compared with WT mice (FIG. 14). Our data suggest that Tollip deficiency may facilitate the resolution of chronic inflammation during AOM-DSS-induced colon tumorigenesis. Other immune cells were further surveyed and did not observe any significant difference in the activation status of B cells, Treg cells, or monocytes in WT versus Tollip-deficient mice subjected to AOM-DSS challenge (FIGS. 13A-13C) Together, these data reveal that Tollip deficiency facilitates the resolution of chronic inflammation during AOM-DSS induced colon tumorigenesis. These findings also support the hypothesis that Tollip−/− neutrophils may enhance tumor-immune defense through facilitating T cell proliferation and activation.

### Tollip Mediates the Immunosuppression of Neutrophils in Suppressing T Cell Proliferation.

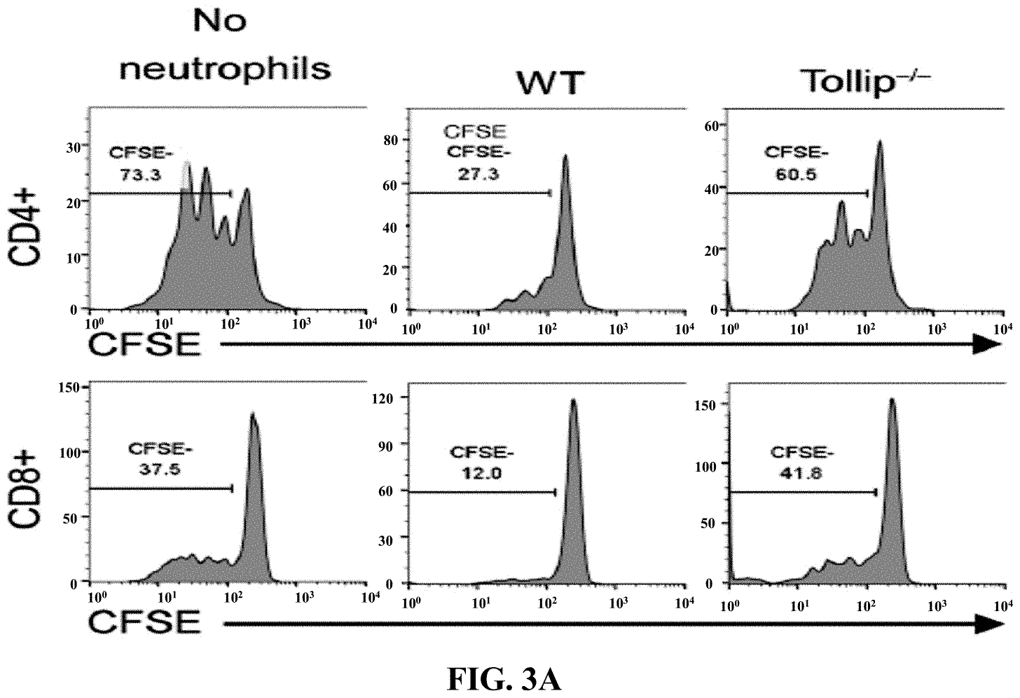

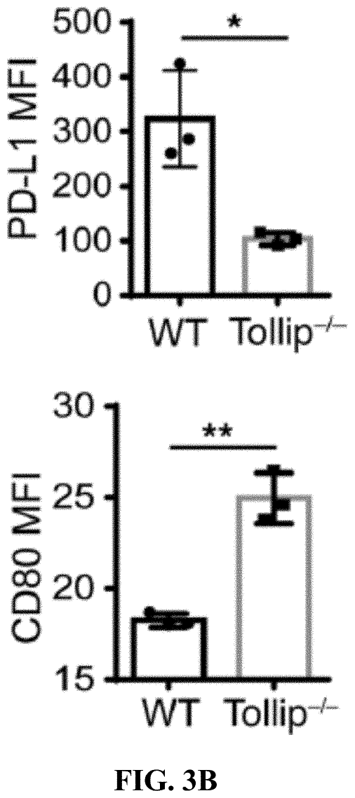

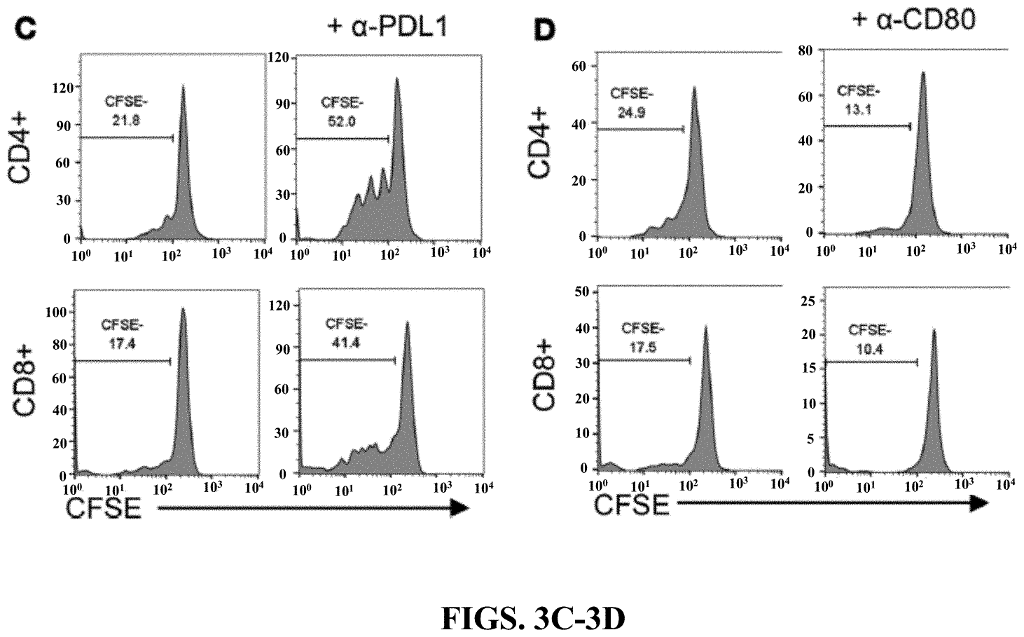

It is well-noticed that tumor-associated neutrophils have immune-suppressive effects by suppressing T cell proliferation (4). To examine that Tollip can facilitate the suppressive effects of neutrophils, in vitro co-culture studies were performed. Increased production of granulocyte macrophage colony-stimulating factor (GM-CSF) has been observed in mucosa of patients with inflammatory bowel disease and rodents subjected to experimental colitis. GM-CSF was also shown to promote the generation of myeloid-derived suppressor cells (12). Bone marrow neutrophils from WT or Tollip−/− mice were cultured in GM-GSF overnight, and subsequently co-cultured with CFSE-labeled allogeneic splenocytes in anti-CD3 coated plates. GM-CSF primed neutrophils showed typical immunosuppressive phenotype, as evident from reduced T cell proliferation with the addition of neutrophils (FIG. 3A). However, compared with WT neutrophils, Tollip−/− neutrophils had significantly less immunosuppressive effects on the proliferation of both CD4 and CD8 T cells (FIG. 3A). Consistent with in vivo results, Tollip−/− neutrophils had increased expression of CD80 and decreased PD-L1 expression (FIG. 3B). To confirm the involvement of PD-L1 or CD80 on Tollip neutrophils during the modulation of T cell proliferation, the blocking antibodies in the co-culture assays were applied. In the presence of anti-PD-L1 antibody, the suppression of WT neutrophils on T cell proliferation was partially removed (FIG. 3C). In contrast, in the presence of anti-CD80 antibody during the co-culture, T cell proliferation were further blocked (FIG. 3D). These data suggest that Tollip−/− neutrophils have reduced suppressive effects on T cell proliferation through an increase of CD80 and a decrease of PD-L1 expression.

### Tollip Deficient Neutrophils Enhance T Cell Activation and Survival

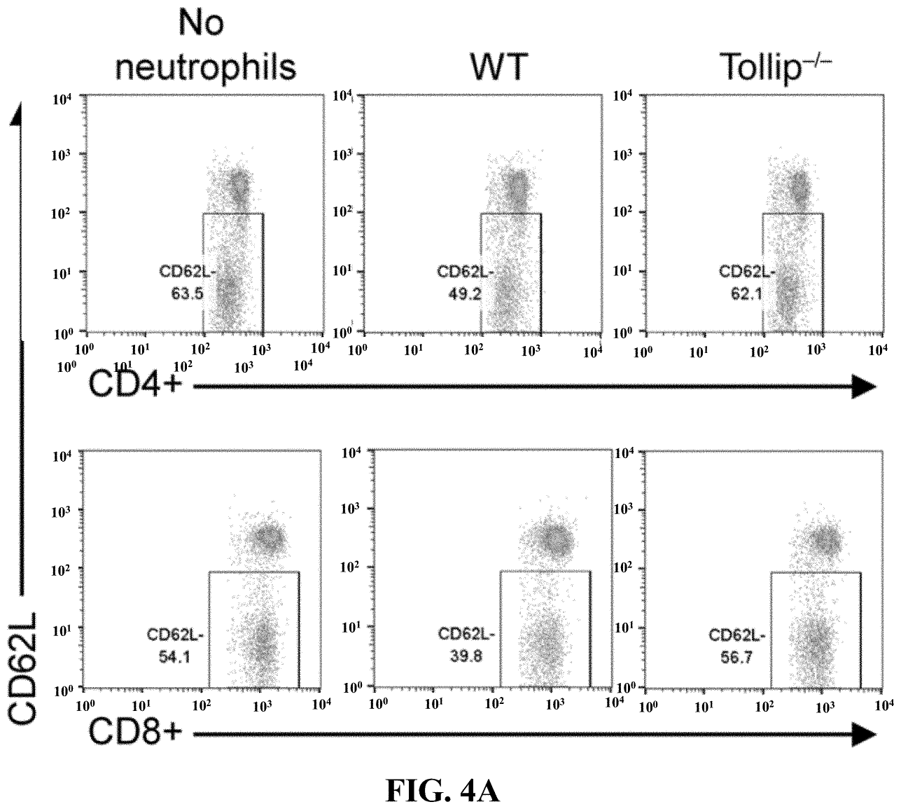

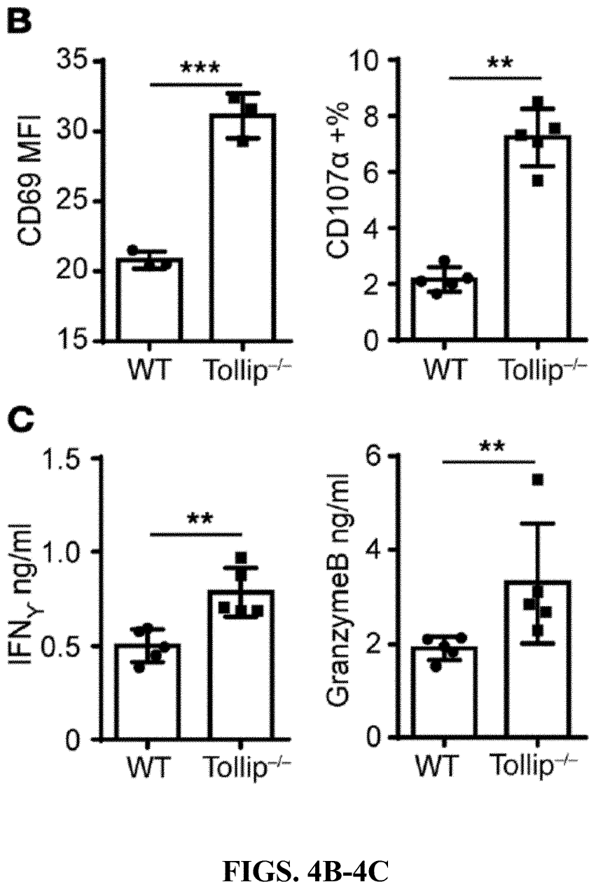

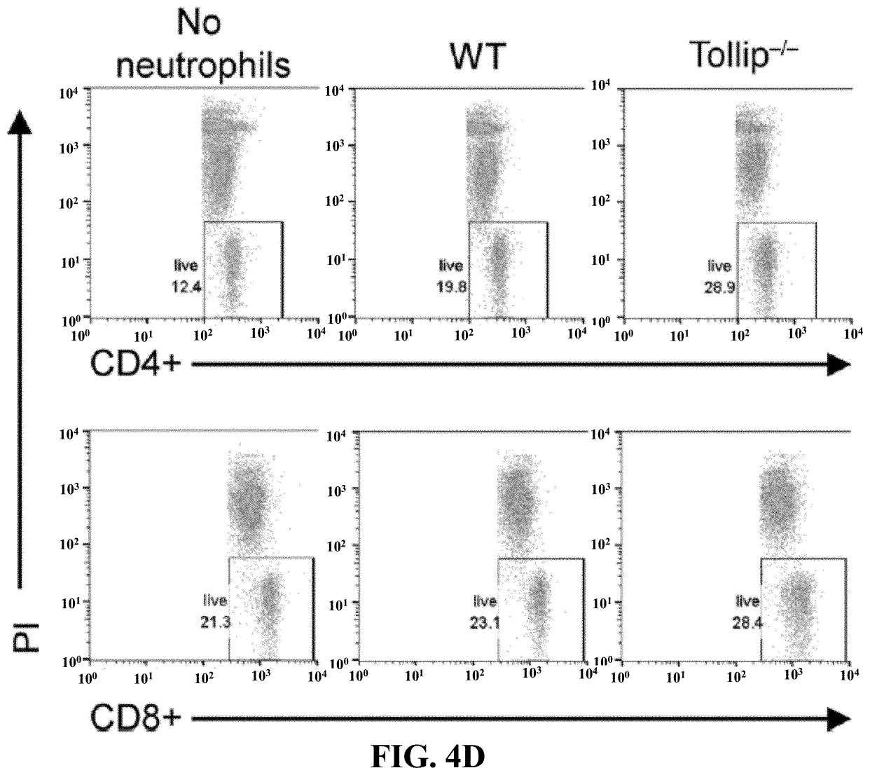

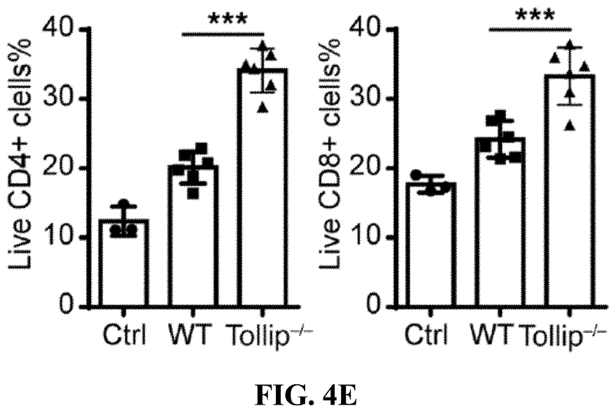

Neutrophils may not only affect the proliferation, but also the survival and activation of T cells. The effects of Tollip−/− neutrophils on T cell activation and survival were measured in vitro through the co-culture assay. For the T cell activation measurement, we tested the surface expression of CD62L and CD107a through flow cytometry, as well as secretants such as INFγ and granzyme B by ELISA. Following 1-day co-culture of GM-CSF-primed neutrophils, both CD4 and CD8 T cells cultured with Tollip−/− neutrophils exhibited significant down-regulation of CD62L as compared to cells co-cultured with WT neutrophils, an indication of enhanced T cell activation (FIG. 4A). The CD69 mean fluorescence intensity (MFI) was significantly increased in CD4+ T cells cocultured with Tollip-deficient neutrophils as compared with WT neutrophils, indicating ele-vated CD4+ T cell activation (FIG. 4B). The populations of CD107α-expressing CD8+ T cells were also significantly elevated upon coculture with Tollip−/− neutrophils as compared with WT neutrophils, indicating elevated CD8+ T cell activation (FIG. 4B). As measured by ELISA, the secreted levels of IFN-γ and gran-zyme B from the cocultures with Tollip−/− neutrophils were significantly higher as compared with the cocul-tures with WT neutrophils (FIG. 4C). Additionally, the application of anti-PD-L1 antibody facilitated the activation of CD4+ T cells and CD8+ T cells (FIGS. 15A-15B). Anti-CD80 antibody also significantly reduced the activation of CD4+ T cells and CD8+ T cells (FIGS. 15A-15B). To assess the effects of Tollip−/− neutrophils on T cell viability, we measured the viability of T cells following a 3-day coculture with either WT or Tollip−/− neutrophils using propidium iodide (PI) staining and flow cytometry. Both CD4+ and CD8+ T cells cocultured with Tollip−/− neutrophils exhibited significantly higher survival rates as compared with T cells cocultured with WT neutrophils (FIGS. 4D and 4E). Collectively, our data reveal that Tollip−/− neutrophils enhance T cell activation as well as survival compared with WT neutrophils.

Tollip Neutrophils have Elevated STAT5 Activation and Reduced STAT1 Activation.

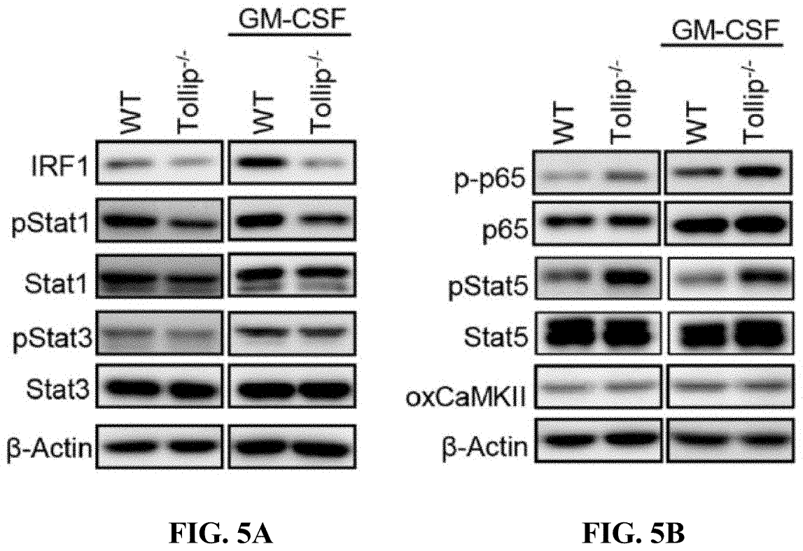

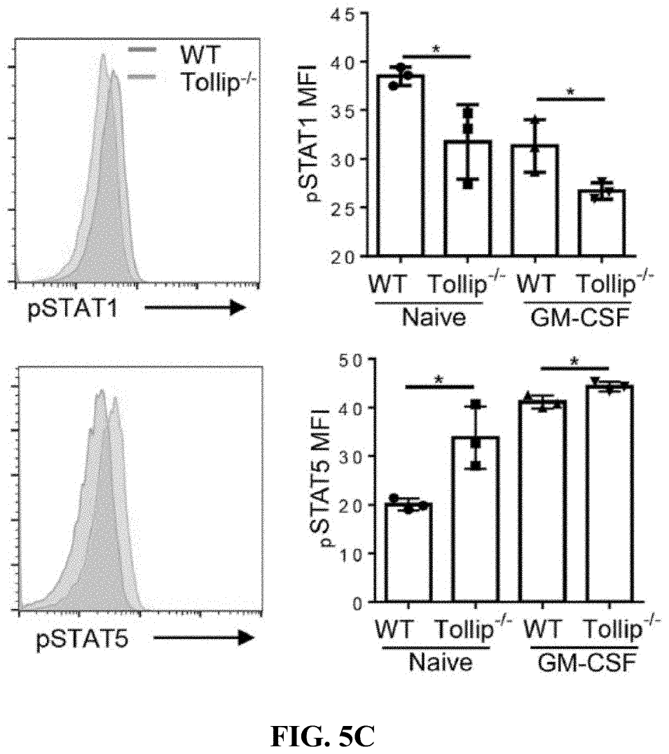

The underlying molecular mechanisms responsible for the neutrophil reprogramming due to Tollip deficiency was examined. Previous studies reported that STAT5 and STAT1 are differentially involved in the expression of CD80 and PD-L1, with STAT5 promoting the expression of CD80 (13) and STAT1/STAT3/IRF1 promoting the expression of PD-L1 (14, 15). Thus, the activation of STAT1, STAT5 was tested as well as other key signaling molecules comparing WT and Tollip−/− neutrophils. It was observed that the phosphorylation levels of STAT1, STAT3 and IRF1 were all reduced in Tollip−/− neutrophils as compared to WT neutrophils (FIG. 5A). This is consistent with reduced PD-L1 expression in Tollip−/− neutrophils. In contrast, an increased phosphorylation of STAT5 and p65 in Tollip−/− neutrophils was detected as compared to WT neutrophils, consistent with elevated expression of CD80 in Tollip−/− neutrophils (FIG. 5B). Elevated oxCAMKII in Tollip−/− neutrophils was observed (FIG. 5C). These mechanistic observations are consistent with elevated CD80 expression in Tollip−/− neutrophils. Correspondingly, quantitative measurement of STAT1/STAT5 phosphorylation through flow cytometry was performed. As shown in FIG. 5B, Tollip neutrophils had significantly elevated levels of p-STAT5 and reduced levels of p-STAT1. These data reveal that the dichotomy of elevated STAT5 activation and reduced STAT1 activation due to Tollip deficiency can underlie the polarized CD80/PD-L1 expression in Tollip deficient neutrophils conducive for an effective T cell response toward tumor immune surveillance.

### Adoptive Transfer of Tollip−/− Neutrophils is Sufficient to Dampen Colitis-Associated Tumor Progression.

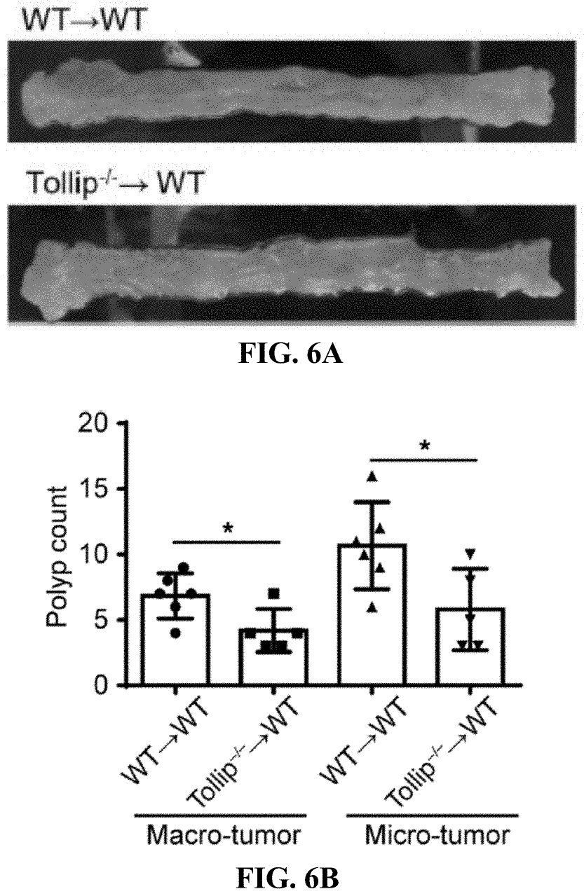

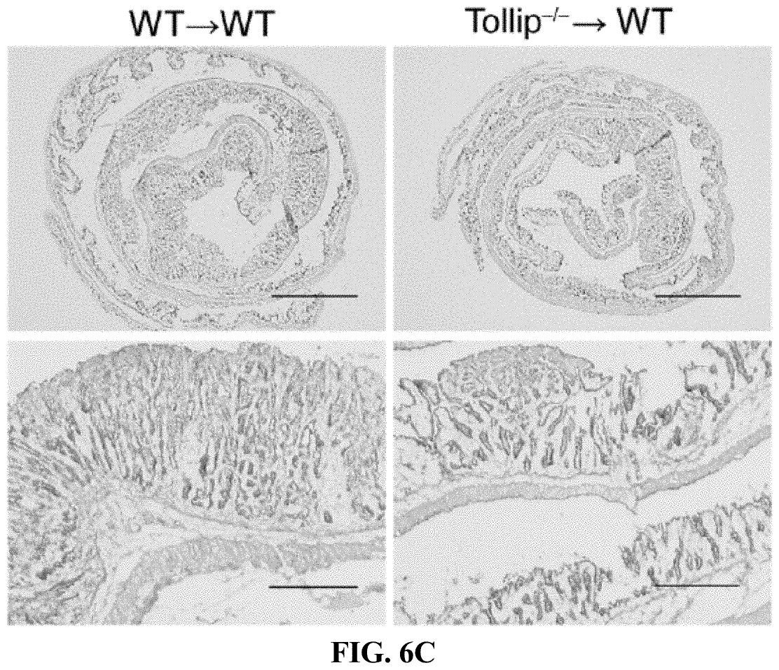

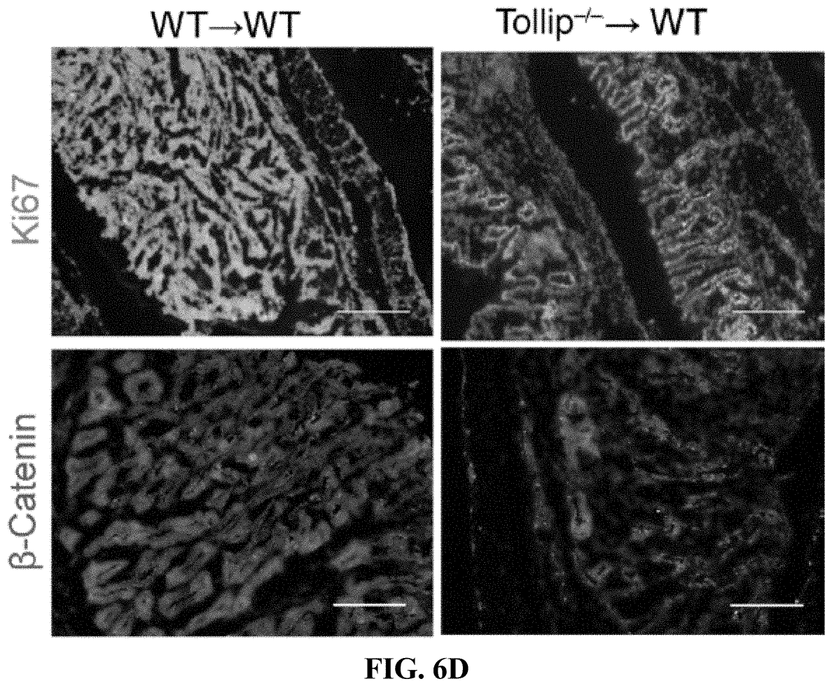

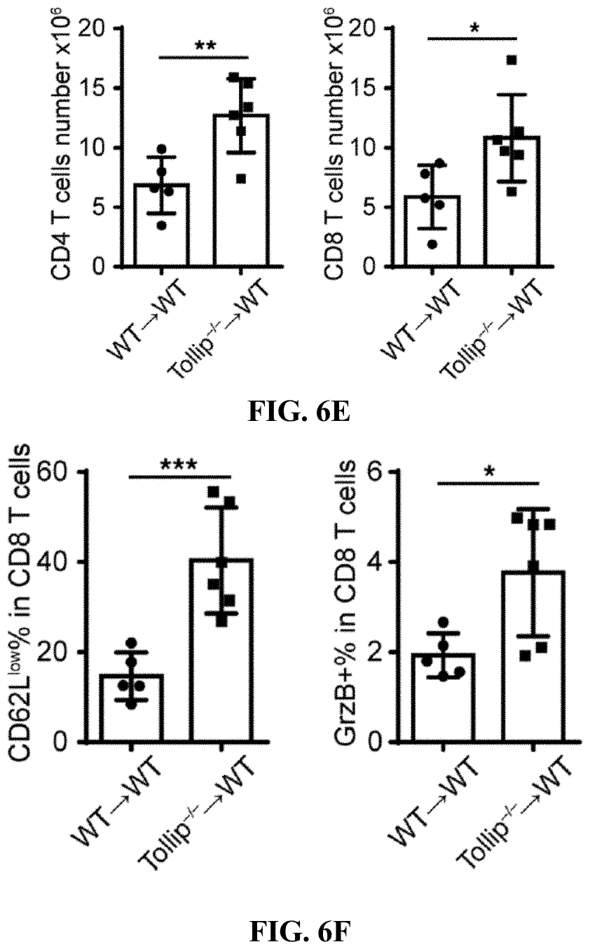

To directly test whether neutrophil deficiency in Tollip is responsible for an effective anti-tumor effect in vivo, the adoptive transfer experiment was next performed. Purified neutrophils from either WT or Tollip−/− mice were injected weekly via i.v. to WT mice subjected to AOM-DSS challenge as described in the Method Section. It was observed that WT mice receiving Tollip−/− neutrophils exhibited a marked reduction in the tumor load as compared to the WT mice receiving WT neutrophils (FIGS. 6A and 6B). The overall body weight as well as colon length were similar among these groups at the end of the study (FIGS. 12A-12D). However, H&E staining revealed reduced inflammation in mice transfused with Tollip−/− neutrophils (FIG. 6C). Ki67 and β-catenin staining revealed that the colons from WT mice transfused with Tollip−/− neutrophils had reduced β-catenin and Ki67 positive cells (FIG. 6D). These data indicate that transfusion of Tollip−/− neutrophils is sufficient to render protection against AOM-DSS induced colon tumorigenesis.

The levels and activation status of CD4 and CD8 T cells in mice transfused with WT or Tollip neutrophils and subjected to the AOM-DSS challenge was examined. As shown in FIG. 6E, mice transfused with Tollip−/− neutrophils had more splenic cell counts of CD4 and CD8 T cells. Furthermore, CD8 T cells in mice transfused with Tollip−/− neutrophils demonstrated significantly elevated activation status as reflected in the higher percentage of CD62Llow as well as Granzyme B positive CD8 T cells (FIG. 6F). These data reveal an enhanced in vivo anti-tumor immunity due to transfusion of Tollip−/− neutrophils.

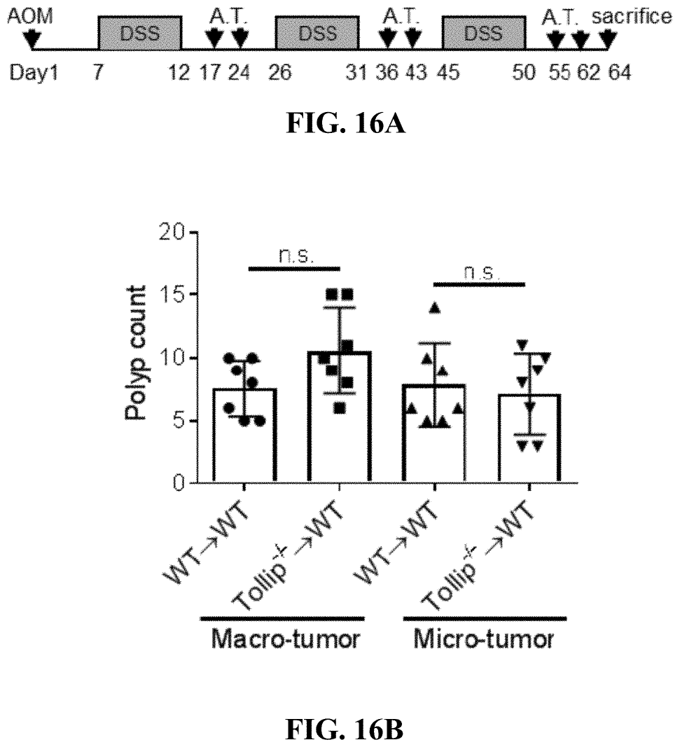

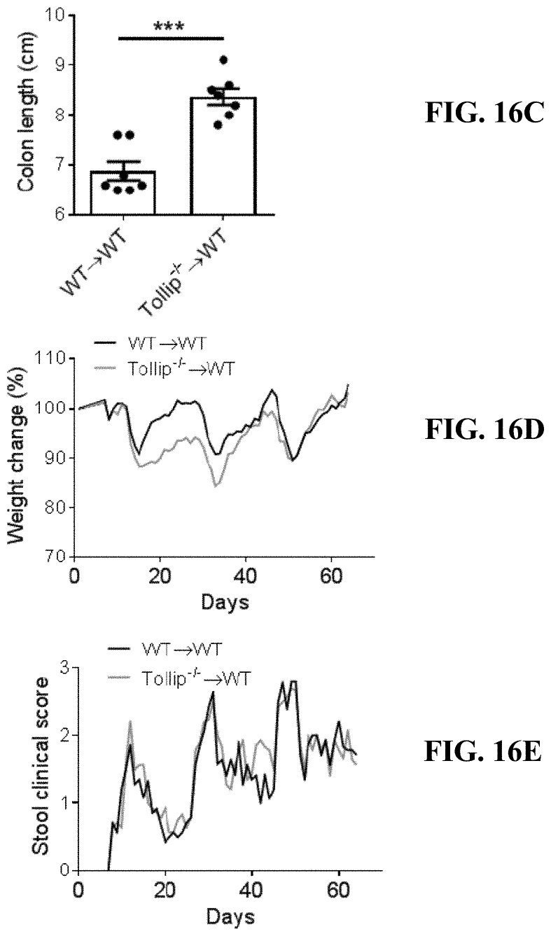

Although the data suggest that reprogrammed neutrophils due to Tollip deficiency exhibit enhanced antitumor immune function in vitro and in vivo, the contribution of other innate leukocytes such as monocytes cannot be excluded. To test whether Tollip-deficient monocytes may have similar effects, an additional adoptive transfer study was performed with WT and Tollip-deficient monocytes. Similar to the neutrophil study, monocytes from either WT or Tollip−/− mice were i.v. injected weekly into WT mice subjected to AOM-DSS challenge (FIGS. 16A-16E). In contrast to the neutrophil transfusion, however, we observed no significant difference in colon tumor burden of recipient mice at the end of the experimental regimen (FIGS. 16A-16E). However, we did observe that mice that received transfusion with Tollip-deficient monocytes exhibited longer colon length as compared with mice transfused with WT monocytes.

### DISCUSSION

This Example can demonstrate that, through Tollip deletion, neutrophils can be uniquely programed to serve as a highly effective anti-tumor immune modulator. Several lines of data support this novel conclusion. First, it was observed that Tollip deficient mice have reduced colon tumor development when subjected to the AOM-DSS challenge. Second, it was observed found that Tollip deficient neutrophils are re-programmed to be conducive for T cell proliferation, survival and activation. Third, it was demonstrated that the transfusion of Tollip deficient neutrophils into WT mice is sufficient to alleviate AOM-DSS induced colon tumor formation.

This data can provide a fresh perspective for the emerging and potentially important roles of neutrophils during the modulation of tumor immune environment. Emerging basic and translational studies with experimental models and human cancer patients suggest complex repertoires of tumor-associated myeloid cells that may either promote or inhibit tumor progression (4). Although tumor tissues are known to have expanded pools of neutrophils that often correlate with aggravated tumor growth (4-6), it is not well understood how tumor-associated neutrophils are programmed at molecular level to either facilitate or suppress tumorigenesis. Earlier studies led to the hypothesis that neutrophils may be differentially activated into either an N1 tumor-promoting state or an N2 tumor-promoting state (16). However, such categorization of neutrophils still lacks phenotypic and mechanistic clarity. The data demonstrated in this Example can extend these emerging studies and provide mechanistic insights regarding the role of neutrophil reprogramming in tumor growth. It was observed in this Example that wild type neutrophils exhibit suppressive function toward T cell proliferation, survival and activation in vitro partly through immune check point PD-L1, resembling the tumor-promoting and T cell-suppressing effects of tumor-associated neutrophils in vivo. In contrast, Tollip−/− neutrophils promote the survival and activation of CD4 and CD8 T cells. Recent studies reveal the significance of innate immune checkpoint molecules such as PD-L1 in suppressing T cell function and promoting tumor immune-evasion (3). This Example can not only confirms the important involvement of PD-L1 and CD80 on neutrophils in modulating T cell function, but also can demonstrate that Tollip deficiency reprograms neutrophils into a T cell-promoting state with significantly reduced PD-L1 and elevated CD80. These data suggest an innovative approach for reprogramming neutrophils through Tollip deletion for enhanced tumor immune-surveillance.

The current study also provides molecular mechanisms responsible for the reprogramming of tumor-suppressing neutrophils. The immune check-point T cell inhibitory molecule PD-L1 expression on neutrophils was shown to be under the control of STAT1, STAT3 and IRF1 (14, 15). It was observed that Tollip deficiency leads to reduced activation of STAT1, STAT3 and IRF1 in Tollip−/− neutrophils that is consistent with reduced PD-L1 expression. On the other hand, STAT5 is responsible for the expression of co-stimulatory molecules such as CD80 on neutrophils (13). The finding that Tollip deficient neutrophils have elevated STAT5 activation provides a mechanistic explanation for elevated CD80 expression.

From a translational perspective, this data reveal a novel potential of using “reprogrammed” innate leukocytes such as neutrophils in tumor treatment. There has been a resurgence in cancer immune-therapy, given the intriguing yet limited success through engineered T cells combined with check-point inhibitors (2). Recent studies also suggest that subsets of neutrophils may hold potential in suppressing cancer cell growth, although underlying mechanisms for the differentiation/activation of tumor-suppressing neutrophils are not clear (4). The adoptive transfer data in this Example with Tollip deficient neutrophils not only provide a proof-of-principle for the translational potential of reprogrammed neutrophils in cancer treatment, but also reveal critical mechanisms for effective reprogramming of tumor-fighting neutrophils through Tollip deletion.

Collectively, this Example can demonstrate that neutrophils engineered with Tollip deletion can be effectively reprogrammed into a novel state to exhibit an effective anti-tumor immune defense. Mechanistically, this Example can demonstrate that Tollip deficient neutrophils can potently activate the functions of both CD4 and CD8 T cells.

### REFERENCES FOR EXAMPLE 1

1. Binnewies M, Roberts E W, Kersten K, Chan V, Fearon D F, Merad M, et al. Understanding the tumor immune microenvironment (TIME) for effective therapy. Nat Med. 2018.
2. Lim W A, and June C H. The Principles of Engineering Immune Cells to Treat Cancer. Cell. 2017; 168(4):724-40.
3. Smyth M J, Ngiow S F, Ribas A, and Teng M W. Combination cancer immunotherapies tailored to the tumour microenvironment. Nat Rev Clin Oncol. 2016; 13(3):143-58.
4. Coffelt S B, Wellenstein M D, and de Visser K E. Neutrophils in cancer: neutral no more. Nature reviews Cancer. 2016; 16(7):431-46.
5. Wang T T, Zhao Y L, Peng L S, Chen N, Chen W, Lv Y P, et al. Tumour-activated neutrophils in gastric cancer foster immune suppression and disease progression through GM-CSF-PD-L1 pathway. Gut. 2017; 66(11):1900-11.
6. Nayak A, McDowell D T, Kellie S J, and Karpelowsky J. Elevated Preoperative Neutrophil-Lymphocyte Ratio is Predictive of a Poorer Prognosis for Pediatric Patients with Solid Tumors. Ann Surg Oncol. 2017; 24(11):3456-62.
7. Powell D R, and Huttenlocher A. Neutrophils in the Tumor Microenvironment. Trends in immunology. 2016; 37(1):41-52.
8. Burns K, Clatworthy J, Martin L, Martinon F, Plumpton C, Maschera B, et al. Tollip, a new component of the IL-1RI pathway, links IRAK to the IL-1 receptor. Nature cell biology. 2000; 2(6):346-51.
9. Lu K, Psakhye I, and Jentsch S. Autophagic Clearance of PolyQ Proteins Mediated by Ubiquitin-Atg8 Adaptors of the Conserved CUET Protein Family. Cell. 2014; 158(3):549-63.
10. Chen K, Yuan R, Zhang Y, Geng S, and Li L. Tollip Deficiency Alters Atherosclerosis and Steatosis by Disrupting Lipophagy. J Am Heart Assoc. 2017; 6(4).
11. Diao N, Zhang Y, Chen K, Yuan R, Lee C, Geng S, et al. Deficiency in Toll-interacting protein (Tollip) skews inflamed yet incompetent innate leukocytes in vivo during DSS-induced septic colitis. Sci Rep. 2016; 6:34672.
12. Gargett T, Christo S N, Hercus T R, Abbas N, Singhal N, Lopez A F, et al. GM-CSF signalling blockade and chemotherapeutic agents act in concert to inhibit the function of myeloid-derived suppressor cells in vitro. Clin Transl Immunology. 2016; 5(12):e119.
13. Tormo A J, and Gauchat J F. A novel role for STAT5 in D C: Controlling the Th2-response. JAKSTAT. 2013; 2(4):e25352.
14. O'Reilly L A, Putoczki T L, Mielke L A, Low J T, Lin A, Preaudet A, et al. Loss of NF-kappaB1 Causes Gastric Cancer with Aberrant Inflammation and Expression of Immune Checkpoint Regulators in a STAT-1-Dependent Manner. Immunity. 2018; 48(3):570-83 e8.
15. Garcia-Diaz A, Shin D S, Moreno B H, Saco J, Escuin-Ordinas H, Rodriguez G A, et al. Interferon Receptor Signaling Pathways Regulating P D-L1 and P D-L2 Expression. Cell reports. 2017; 19(6):1189-201.
16. Shaul M E, and Fridlender Z G. Neutrophils as active regulators of the immune system in the tumor microenvironment. Journal of leukocyte biology. 2017; 102(2):343-9.
17. Chen K, Geng S, Yuan R, Diao N, Upchurch Z, and Li L. Super-low dose endotoxin pre-conditioning exacerbates sepsis mortality. EBioMedicine. 2015; 2(4):324-33.
18. Geng S, Chen K, Yuan R, Peng L, Maitra U, Diao N, et al. The persistence of low-grade inflammatory monocytes contributes to aggravated atherosclerosis. Nature communications. 2016; 7:13436.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:

1. A modified neutrophil comprising: reduced or eliminated Tollip gene expression and/or amount of Tollip protein as compared to a wild-type or suitable control neutrophil.

2. The modified neutrophil of aspect 1, wherein the modified neutrophil comprises a deletion of one or more copies of the Tollip gene.

3. The modified neutrophil of aspect 1, wherein the modified neutrophil comprises a Tollip gene silencing oligonucleotide.

4. The modified neutrophil of any one of aspects 1-3, further comprising a suicide gene.

5. The modified neutrophil of any one of aspects 1-4, wherein the modified neutrophil has increased gene and/or protein expression of CD80 as compared to a wild-type neutrophil or suitable control.

6. The modified neutrophil of any one of aspects 1-5, wherein the modified neutrophil has decreased gene and/or protein expression of PDL-las compared to a wild-type neutrophil or a suitable control.

7. The modified neutrophil of any one of aspects 1-6, wherein the modified neutrophil is a human neutrophil.

8. A pharmaceutical formulation comprising:
a modified neutrophil as in any one of aspects 1-7 or a population thereof; and
a pharmaceutically acceptable carrier.

9. The pharmaceutical formulation of aspect 8, wherein the pharmaceutical formulation comprises a therapeutically effective amount of the modified neutrophil or population thereof.

10. A method of generating a Tollip deficient neutrophil or population thereof, the method comprising:
harvesting neutrophils from a subject to obtain harvested neutrophils;
deleting one or more copies of the Tollip gene in one or more of the harvested neutrophils in vitro to obtain the Tollip deficient neutrophil or population thereof.

11. The method of aspect 10, wherein the Tollip deficient neutrophil or population thereof has reduced or eliminated Tollip gene expression and/or amount of Tollip protein as compared to a wild-type or suitable control neutrophil.

12. The method of any one of aspects 10-11, wherein the Tollip deficient neutrophil or population thereof has increased gene and/or protein expression of CD80 as compared to a wild-type neutrophil or suitable control.

13. The method of any one of aspects 10-12, wherein the Tollip deficient neutrophil or population thereof has decreased gene and/or protein expression of PDL-las compared to a wild-type neutrophil or suitable control.

14. The method of any one of aspects 10-13, wherein the method further comprises the step of transforming a harvested neutrophil or the Tollip deficient neutrophil or population thereof to contain and/or conditionally express a suicide gene.

15. The method of any one of aspects 10-14, wherein the subject is human.

16. The method of any one of aspects 10-15, further comprising the step of administering the Tollip deficient neutrophil or population thereof to a subject in need thereof.

17. The method of aspects 16, wherein the subject and the subject in need thereof are the same.

18. The method of aspect 16, wherein the subject and the subject in need thereof are the different.

19. A method of generating a Tollip deficient neutrophil or population thereof, the method comprising: transforming a neutrophil with a Tollip gene silencing oligonucleotide to generate the Tollip deficient neutrophil or population thereof.

20. The method of aspect 19, further comprising the step of transforming a neutrophil with a suicide gene.

21. The method of any one of aspects 19-20, wherein the method further comprises harvesting neutrophils from a subject to obtain harvested neutrophils and wherein the one or more of the harvested neutrophils are transformed in vitro to generate a Tollip deficient neutrophil or population thereof.

22. The method of any one of aspects 19-21, further comprising administering the Tollip deficient neutrophil or population thereof to a subject in need thereof.

23. The method of aspect 22, wherein the subject and the subject in need thereof are the same.

24. The method of aspect 22, wherein the subject and the subject in need thereof are different.

25. The method of any one of aspects 21-24, wherein the subject and the subject in need thereof are human.

26. The method of aspect 19, wherein the method further comprises administering a Tollip gene silencing oligonucleotide to a subject in need thereof.

27. The method of aspect 26, wherein the step of transformation occurs in vivo.

28. The method of any one of aspects 26-27, wherein the subject in need thereof is human.

29. The method of any one of aspects 19-28, wherein the Tollip deficient neutrophil or population thereof has reduced or eliminated Tollip gene expression and/or amount of Tollip protein as compared to a wild-type or suitable control neutrophil.

30. The method of any one of aspects 19-29, wherein the Tollip deficient neutrophil or population thereof has increased gene and/or protein expression of CD80 as compared to a wild-type neutrophil or suitable control.

31. The method of any one of aspects 19-30, wherein the Tollip deficient neutrophil or population thereof has decreased gene and/or protein expression of PDL-las compared to a wild-type neutrophil or suitable control.

32. A method comprising:

administering a modified neutrophil or population thereof as in any one of aspects 1-7 to a subject.

33. The method of aspect 32, wherein the subject is a subject in need thereof and has or is suspected of having a cancer.

34. A method comprising: administering a pharmaceutical formulation as in any one of aspects 8-9 to a subject.

35. The method of aspect 34, wherein the subject is a subject in need thereof and has or is suspected of having a cancer.

36. A method of treating and/or preventing cancer in a subject in need thereof, the method comprising: administering a modified neutrophil or population thereof as in any one of aspects 1-7 to the subject in need thereof.

37. A method of treating and/or preventing cancer in a subject in need thereof, the method comprising: administering a pharmaceutical formulation as in any one of aspects 8-9 to the subject in need thereof.

38. A method of treating and/or preventing cancer in a subject in need thereof, the method comprising: administering a Tollip gene silencing oligonucleotide to a subject in need thereof.

What is claimed is:

1. A modified neutrophil comprising:
reduced or eliminated Tollip gene expression and/or amount of Tollip protein as compared to a wild-type or suitable control neutrophil.

2. The modified neutrophil of claim 1, wherein the modified neutrophil comprises a deletion of one or more copies of the Tollip gene.

3. The modified neutrophil of claim 1, wherein the modified neutrophil comprises a Tollip gene silencing oligonucleotide.

4. The modified neutrophil of claim 1, further comprising a suicide gene.

5. The modified neutrophil of claim 1, wherein the modified neutrophil has increased gene and/or protein expression of CD80 as compared to a wild-type neutrophil or suitable control.

6. The modified neutrophil of claim 1, wherein the modified neutrophil has decreased gene and/or protein expression of PDL-las compared to a wild-type neutrophil or a suitable control.

7. The modified neutrophil of claim 1, wherein the modified neutrophil is a human neutrophil.

8. A pharmaceutical formulation comprising:
a modified neutrophil as in any one of claims 1-7 or a population thereof; and
a pharmaceutically acceptable carrier.

9. The pharmaceutical formulation of claim 8, wherein the pharmaceutical formulation comprises a therapeutically effective amount of the modified neutrophil or population thereof.

10. A method of generating a Tollip deficient neutrophil or population thereof, the method comprising:
harvesting neutrophils from a subject to obtain harvested neutrophils;
deleting one or more copies of the Tollip gene in one or more of the harvested neutrophils in vitro to obtain the Tollip deficient neutrophil or population thereof.

11. The method of claim 10, wherein the Tollip deficient neutrophil or population thereof has reduced or eliminated Tollip gene expression and/or amount of Tollip protein as compared to a wild-type or suitable control neutrophil.

12. The method of claim 10, wherein the Tollip deficient neutrophil or population thereof has increased gene and/or protein expression of CD80 as compared to a wild-type neutrophil or suitable control.

13. The method of claim 10 or 12, wherein the Tollip deficient neutrophil or population thereof has decreased gene and/or protein expression of PDL-las compared to a wild-type neutrophil or suitable control.

14. The method of claim 10, wherein the method further comprises the step of transforming a harvested neutrophil or the Tollip deficient neutrophil or population thereof to contain and/or conditionally express a suicide gene.

15. The method of claim 10, wherein the subject is human.

16. The method of claim 10, further comprising the step of administering the Tollip deficient neutrophil or population thereof to a subject in need thereof.

17. The method of claim 16, wherein the subject and the subject in need thereof are the same.

18. The method of claim 16, wherein the subject and the subject in need thereof are the different.

19. A method of generating a Tollip deficient neutrophil or population thereof, the method comprising:
transforming a neutrophil with a Tollip gene silencing oligonucleotide to generate the Tollip deficient neutrophil or population thereof.

20. The method of claim 19, further comprising the step of transforming a neutrophil with a suicide gene.

21. The method of claim 19, wherein the method further comprises harvesting neutrophils from a subject to obtain harvested neutrophils and wherein the one or more of the harvested neutrophils are transformed in vitro to generate a Tollip deficient neutrophil or population thereof.

22. The method of claim 19, further comprising administering the Tollip deficient neutrophil or population thereof to a subject in need thereof.

23. The method of claim 22, wherein the subject and the subject in need thereof are the same.

24. The method of claim 22, wherein the subject and the subject in need thereof are different.

25. The method of claim 21, wherein the subject and the subject in need thereof are human.

26. The method of claim 19, wherein the method further comprises administering a Tollip gene silencing oligonucleotide to a subject in need thereof.

27. The method of claim 26, wherein the step of transformation occurs in vivo.

28. The method of claim 26, wherein the subject in need thereof is human.

29. The method of claim 19, wherein the Tollip deficient neutrophil or population thereof has reduced or eliminated Tollip gene expression and/or amount of Tollip protein as compared to a wild-type or suitable control neutrophil.

30. The method of claim 19, wherein the Tollip deficient neutrophil or population thereof has increased gene and/or protein expression of CD80 as compared to a wild-type neutrophil or suitable control.

31. The method of claim 19, wherein the Tollip deficient neutrophil or population thereof has decreased gene and/or protein expression of PDL-las compared to a wild-type neutrophil or suitable control.

32. A method comprising:
administering a modified neutrophil or population thereof as in any one of claims 1-7 to a subject.

33. The method of claim 32, wherein the subject is a subject in need thereof and has or is suspected of having a cancer.

34. A method comprising:
administering a pharmaceutical formulation as in claim 8 to a subject.

35. The method of claim 34, wherein the subject is a subject in need thereof and has or is suspected of having a cancer.

36. A method of treating and/or preventing cancer in a subject in need thereof, the method comprising:
administering a modified neutrophil or population thereof as in any one of claims 1-7 to the subject in need thereof.

37. A method of treating and/or preventing cancer in a subject in need thereof, the method comprising:
administering a pharmaceutical formulation as in claim 8 to the subject in need thereof.

38. A method of treating and/or preventing cancer in a subject in need thereof, the method comprising:
administering a Tollip gene silencing oligonucleotide to a subject in need thereof.
